(19) United States
(12) Patent Application Publication
(10) Pub. No.: US 2024/0341889 A1
(43) Pub. Date: Oct. 17, 2024

(54) SURGERY ASSISTING DEVICE

(71) Applicant: RIVERFIELD Inc., Tokyo (JP)

(72) Inventors: Masao KANAZAWA, Tokyo (JP); Kotaro TADANO, Tokyo (JP)

(73) Assignee: RIVERFIELD Inc., Tokyo (JP)

(21) Appl. No.: 18/754,314

(22) Filed: Jun. 26, 2024

Related U.S. Application Data
(63) Continuation of application No. PCT/JP2021/048580, filed on Dec. 27, 2021.

(51) Int. Cl. A61B 34/00; A61B 34/37

(52) U.S. Cl. CPC A61B 34/74; A61B 34/25; A61B 34/37

(57) ABSTRACT

A master-side device for remotely manipulating a slave-side device includes a first manipulator used to manipulate a movable part of the slave-side device, and control processing circuitry that performs determining processing of determining whether or not to turn the movable part in a roll direction, and based on determining to turn the movable part in the roll direction, control the movable part to turn in the roll direction in response to a specific manipulation of the first manipulator.

### CROSS-REFERENCE TO RELATED APPLICATION

This Application is a Continuation of International Application No. PCT/JP2021/048580, filed Dec. 27, 2021 in the Japanese Patent Office, the contents of which being herein incorporated by reference in its entirety.

### BACKGROUND

The present disclosure relates to a technique for a surgery assisting device, and particularly to a technique for a surgery assisting device in which a master-side device and a slave-side device are linked to perform surgery on a patient.

In related art surgery assistance devices, a surgery proceeds with linking the movements of the surgeon's hands to the movements of surgical instruments such as forceps placed on the patient's side to cause the surgical instruments to move as intended by the surgeon.

However, the range of movement of the surgeon's hand is limited, and in some cases, the surgical instrument may not be able to be moved as intended.

### SUMMARY

It is an aspect to provide a surgery assisting device that can perform surgery appropriately.

According to an aspect of one or more embodiments, there is provided a master-side device for remotely manipulating a slave-side device, the master-side device comprising a first manipulator used to manipulate a movable part of the slave-side device; and control processing circuitry configured to at least perform determining processing of determining whether or not to turn the movable part in a roll direction, and based on determining to turn the movable part in the roll direction, control the movable part to turn in the roll direction in response to a specific manipulation of the first manipulator.

According to another aspect of one or more embodiments, there is provide a master-side device for remotely manipulating a slave-side device, the master-side device comprising a first manipulator having a range of movement in a roll direction, the first manipulator manipulating a movable part of the slave-side device; and control processing circuitry configured to at least determine whether or not to turn the movable part in the roll direction, and based on determining to turn the movable part in the roll direction, control the movable part to turn in the roll direction in response to a roll manipulation of the first manipulator.

According to yet another aspect of one or more embodiments, there is provided master-side device for remotely manipulating a slave-side device, the master-side device comprising a display; a first manipulator that has a linkage with the slave-side device, the linkage allowing the first manipulator to manipulate a movable part of the slave-side device; and control processing circuitry configured to at least control the display to display a button that toggles between controlling to turn the movable part in a roll direction and not controlling to turn the movable part in the roll direction in response to a roll manipulation of the first manipulator, and based on a first state of the button, maintain the linkage such that the movable part of the slave-side device is turned in response to the roll manipulation of the first manipulator, and based on a second state of the button, switch off the linkage such that when the first manipulator is turned in the roll direction in response to the roll manipulation the movable part of the slave-side device is not turned in the roll direction.

### BRIEF DESCRIPTION OF DRAWINGS

FIG. 1 is a perspective view of a master-side device according to some embodiments.

FIG. 2 is a perspective view of a slave-side device, according to some embodiments.

FIG. 3 is a perspective view of a right manipulator, according to some embodiments.

FIG. 4 is a perspective view of a left manipulator, according to some embodiments.

FIG. 5 is a perspective view illustrating a state (reference position) in which each movable part of a delta structure is located approximately at the center in the corresponding range of movement, according to some embodiments.

FIG. 6 is a perspective view illustrating a state in which a support part of the delta structure is located forward (on the surgeon's side) of the reference position, according to some embodiments.

FIG. 7 is a perspective view illustrating a state in which the support part of the delta structure is located above the reference position, according to some embodiments.

FIG. 8 is a perspective view conceptually illustrating an attitude changing structure, according to some embodiments.

FIG. 9 is a perspective view schematically illustrating the movement of the attitude changing structure in a turning manipulation in the pan direction, according to some embodiments.

FIG. 10 is a perspective view schematically illustrating the movement of the attitude changing structure in a turning manipulation in the pitch direction, according to some embodiments.

FIG. 11 is a perspective view schematically illustrating the movement of the attitude changing structure in a turning manipulation in the roll direction, according to some embodiments.

FIG. 12 is a side view mainly illustrating a grasping structure of the attitude changing structure, according to some embodiments.

FIG. 13 is a cross-sectional view illustrating a trocar placed on an abdominal wall of a patient and a surgical instrument inserted into the trocar, according to some embodiments.

FIG. 14 is a functional block diagram of a surgery assisting system, according to some embodiments.

FIG. 15 illustrates a state in which a first setting tab is selected on a setting screen displayed on a second monitor, according to some embodiments.

FIG. 16 illustrates a state in which a second setting tab is selected on the setting screen displayed on the second monitor, according to some embodiments.

FIG. 17 is an example of a flowchart for implementing a neutral assistance function, according to some embodiments.

FIG. 18 is an example of a flowchart for implementing a wrist angle scaling function, according to some embodiments.

FIG. 19 is an example of a flowchart for implementing a roll clutch function, according to some embodiments.

FIG. 20 illustrates a modification example of a state in which the second setting tab is selected on the setting screen displayed on the second monitor, according to some embodiments.

### DETAILED DESCRIPTION

A surgery assisting device according to various embodiments may be a master-side device for remotely manipulating a slave-side device, and may include a first manipulator having a range of movement and used to manipulate a movable part of the slave-side device; and a control unit configured to control the movable part to turn in a roll direction in response to a specific manipulation on the first manipulator, wherein the control unit performs processing of determining where or not to turn the movable part in the roll direction in response to the specific manipulation.

This configuration makes it possible to remove the linkage between the master-side device 1 and the slave-side device 2. In some embodiments, a configuration is provided in which the linkage can be removed only for translational motion in a direction or a turning motion in a direction around an axis, making it possible to provide manipulation modes that meet various needs of the surgeon.

According to various embodiments, it is possible to provide a surgery assisting device that can perform surgery appropriately.

### 1. Configuration of Surgery Assisting System

A surgery assisting system S according to an embodiment will be described below with reference to the attached drawings.

Note that each configuration described in the drawings and referred to herein is merely an example for realizing various embodiments of the present disclosure. Therefore, various changes can be made to the various embodiments without departing from the technical idea of the present disclosure. Further, in order to avoid duplicate explanation, configurations that have been described once may be given the same reference numerals and repeated explanations may be omitted for conciseness.

The surgery assisting system S includes a master-side device 1 and a slave-side device 2.

The master-side device 1 is a device used by a surgeon (doctor) who performs surgery. Further, the slave-side device 2 is a device that is installed in an operating room where a patient to be operated on is lying down, and performs an actual surgery on the patient.

The master-side device 1 and the slave-side device 2 are installed apart from each other. The master-side device 1 and the slave-side device 2 may be installed in separate rooms, or may be installed in the same room.

The master-side device 1 and the slave-side device 2 are connected to each other to enable wired or wireless data communication between the master-side device 1 and the slave-side device 2, and each part of the remotely located slave-side device 2 is driven to perform surgery on the patient in accordance with a surgeon's manipulation on the master-side device 1.

In the various embodiments described below, an example is described in which the master-side device 1 and the slave-side device 2 are installed and used on the floor of an operating room. However, the scope of the present disclosure is not limited to the type that is installed and used on the floor of an operating room, and various embodiments may also be applied to a configuration in which the master-side device 1 and the slave-side device 2 are attached to and used on the ceiling or wall of an operating room.

Note that the front and back, up and down, and left and right directions described below are for convenience of explanation, and the implementation of the various embodiments is not limited by these directions.

Further, the surgery assisting system S described below has a configuration in which a movable body turns, but this “turning” means a movement in the direction around a reference axis (central axis) that includes any one point, and refers to a motion that also includes “rotating”. Note that although an example is described below in which a movable body turns, the movable body is not limited to performing a turning motion, and may also perform a straight (e.g., linear) motion, and the term “motion” includes within its scope both a turning motion and a straight (e.g., linear) motion.

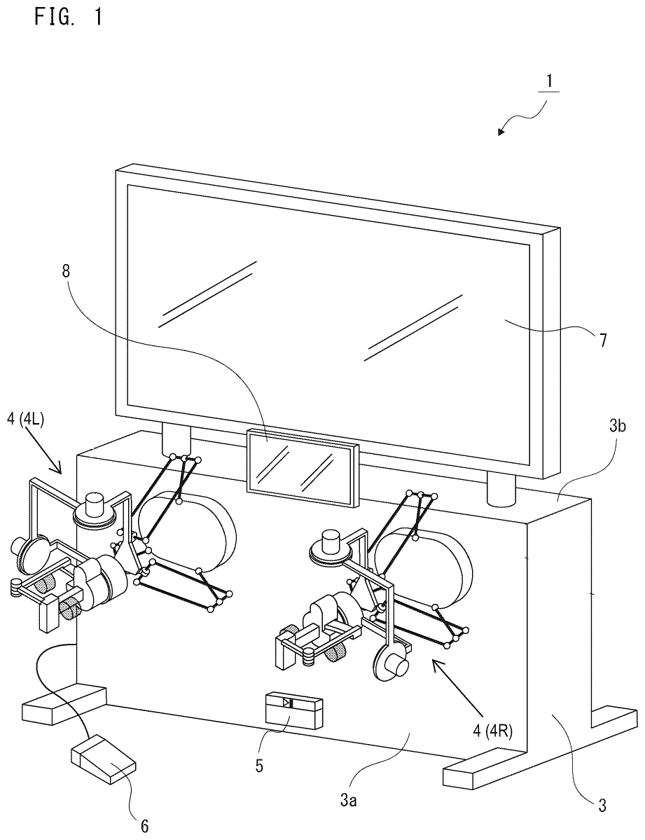

A specific configuration of the master-side device 1 is illustrated in FIG. 1.

Note that in the explanation of the master-side device 1, the front and back, and left and right directions are described supposing that the direction of the surgeon viewed from the master-side device 1 is the front. In other words, the surgeon is located in front of the master-side device 1.

The master-side device 1 includes a base part 3 that is installed on the floor, two manipulators 4 that are attached separated apart from each other in the left and right direction above a front face part 3a of the base part 3, a first foot button 5 that is attached at the center below the front face part 3a, a second foot button 6 that is connected to the base part 3 by wire and can be placed on the floor, a first monitor 7 that is attached to a top face part 3b of the base part 3, and a second monitor 8 that is attached to the top face part 3b and is smaller than the first monitor 7.

One of the two manipulators 4 is a right manipulator 4R that is handled by the surgeon's right hand, and the other is a left manipulator 4L that is handled by the surgeon's left hand.

The surgeon manipulates the right manipulator 4R with the right hand and the left manipulator 4L with the left hand while sitting on a chair (not illustrated) placed in front of the base part 3 (on the front face part 3a side).

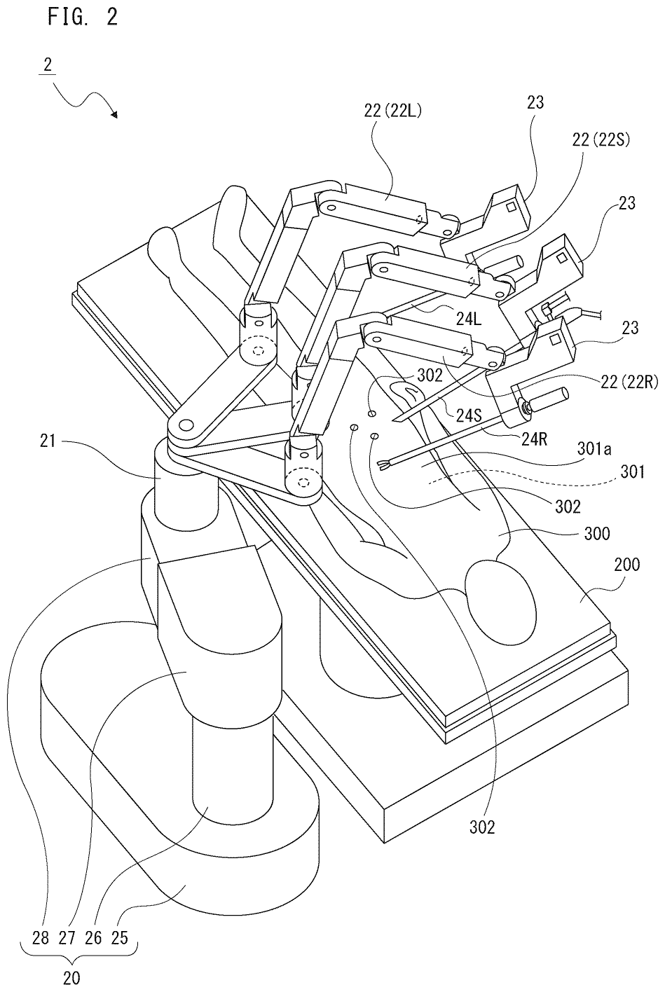

In response to manipulations using the right manipulator 4R and the left manipulator 4L, the slave-side device 2 illustrated in FIG. 2 is driven to perform surgery on the patient. The specific structures and manipulations of the right manipulator 4R and the left manipulator 4L, and the operation of the slave-side device 2 in response to the manipulations will be described later.

The first foot button 5 is configured to switch whether or not to link a manipulation on the master-side device 1 with the operation of the slave-side device 2. Since the surgeon uses both hands to manipulate the manipulators 4, it is advantageous that the first foot button 5 be installed at a position where the surgeon can manipulate the first foot button 5 with the foot.

Each time the first foot button 5 is manipulated, the master-side device 1 and the slave-side device 2 are switched between a state in which the master-side device 1 and the slave-side device 2 are linked and a state in which the master-side device 1 and the slave-side device 2 are not linked.

Note that the state in which both the master-side device 1 and the slave-side device 2 are linked refers to a state in which each part of the slave-side device 2, such as forceps, is driven in response to a manipulation on the manipulators 4 of the master-side device 1. Further, the state in which both the master-side device 1 and the slave-side device 2 are not linked refers to a state in which each part of the slave-side device 2, such as forceps, is not driven and the position and attitude of each part are maintained even when the manipulators 4 of the master-side device 1 are manipulated.

In an embodiment, the second foot button 6 may be configured to capture an image of a surgical field, such as the inside of the patient's body cavity. In an embodiment, the second foot button 6 may be an image capture button. For example, in some embodiments, the slave-side device 2 may include an endoscope in addition to the forceps manipulated by the right manipulator 4R and the forceps manipulated by the left manipulator 4L. The second foot button 6, when actuated, may capture and save an image based on the viewing angle of the endoscope.

The first monitor 7 may be a monitor device for displaying a live view image through the endoscope included in the slave-side device 2.

The second monitor 8 may be a monitor device for displaying various types of information, and can display attitude information of the endoscope and various setting screens, for example. Screens to be displayed on the second monitor 8 will be described later.

The first monitor 7 and the second monitor 8 may each be, for example, a display device such as a liquid crystal display (LCD) or an organic electro-luminescence (EL) display.

A general configuration of the slave-side device 2 will be described with reference to FIG. 2. An operating table 200 is installed in the operating room, and a patient 300 is lying on the operating table 200, for example, in a supine state. One or more ports 302 are formed in a portion forming a body cavity 301 of the patient 300, for example, an abdominal wall 301a. A part (distal end part) of a surgical instrument, which will be described later, is inserted into each port 302 when a surgical operation is performed. The port 302 is a small hole into which a shaft-shaped surgical instrument is inserted.

The slave-side device 2 includes a base 20 that is placed on the floor or the like of the operating room, a stage 21 that is attached to the base 20, one or more arms 22 that are attached to the stage 21, surgical instrument holding devices 23 that are attached to the distal ends of the arms 22, respectively, and surgical instruments 24 that are detachably held by the surgical instrument holding devices 23, respectively.

The base 20 includes a base part 25 that is placed on the floor or the like of the operating room, a lifting mechanism 26 that is attached to the top of the base part 25, a rear base arm 27 that is attached to the top of the lifting mechanism 26, and a front base arm 28 that is attached to the rear base arm 27 so as to be extendable therefrom in the horizontal direction.

The lifting mechanism 26 extends and contracts in the vertical direction to elevate and lower the rear base arm 27 in the vertical direction so that the rear base arm 27 can be adjusted to an appropriate height position.

The front base arm 28 can extend from the rear base arm 27 in the horizontal direction so that the position of the stage 21 attached to the distal end of the front base arm 28 can be adjusted.

The stage 21 is formed on the front base arm 28 to pivotally support the arms 22.

In the embodiment illustrated in FIG. 2, three arms 22 are attached as the arms 22. One arm is a right arm 22R that holds a surgical instrument 24R that is driven in response to a manipulation of the right manipulator 4R of the master-side device 1, another arm is a left arm 22L that holds a surgical instrument 24L that is driven in response to a manipulation of the left manipulator 4L of the master-side device 1, and the remaining arm 22S is an endoscope arm 22S that holds an endoscope 24S as the surgical instrument 24. Note that although a configuration is illustrated by way example in which the endoscope 24S is provided as the surgical instrument 24, one of the surgical instruments 24 may be an imaging device other than an endoscope.

In the following description, when the term “surgical instrument 24” is used without specific explanation, the term “surgical instrument 24” includes within its scope not only the surgical instruments 24R and 24L such as forceps but also the endoscope 24S. That is, the surgical instrument 24 may include, for example, a forceps, an endoscope, and/or any device used for surgery on a patient.

Each arm 22 is turnable in a direction around an axis extending vertically on the stage 21.

Each arm 22 includes one or more joints and turning parts, and has a mechanism for easily moving a distal end of the arm 22 to any position.

The surgical instrument holding device 23 is attached to the distal end of the arm 22 through a gimbal or the like.

In some embodiments, the endoscope 24S may be, for example, provided as an endoscope device having an endoscope, and may include a shaft extending back and forth, a camera head connected to the distal end part of the shaft, and a light guide connected to the middle part of the shaft.

The distal end of the shaft of the surgical instrument 24 may be inserted into the body cavity 301 through any one of the ports 302 formed in the patient 300.

In a state in which the distal end of the shaft is inserted into the body cavity 301, illumination light is emitted from the distal end of the shaft of the endoscope device, and the state inside the body cavity 301 is captured by an imaging element. In an embodiment, the acquisition of an image based on the capture by the imaging element is executed, for example, by the second foot button 6 illustrated in FIG. 1.

The internal state of the body cavity 301 captured by the image sensor is transmitted as a captured image signal to the first monitor 7 illustrated in FIG. 1, making it possible for the surgeon to remotely observe the internal state of the body cavity 301.

The surgeon can remotely manipulate the position and attitude of the surgical instrument 24R attached to the right arm 22R by manipulating the right manipulator 4R. Further, the surgeon can remotely manipulate the position and attitude of the surgical instrument 24L attached to the left arm 22L by manipulating the left manipulator 4L.

All or some of movable parts such as joints and turning parts of each arm 22 are driven by actuators such as built-in motors in response to a remote manipulation using the master-side device 1.

Note that although the slave-side device 2 is illustrated by way of example in which three surgical instruments 24 can be manipulated by the three arms 22, considering that the slave-side device 2 is used for surgery by a remote manipulation using the master-side device 1, any device may be used as long as the device can handle the endoscope 24S and other surgical instruments 24 with two arms.

Thus, according to various embodiments, the surgical instruments 24 other than the endoscope 24S may be remotely manipulated by the right manipulator 4R, remotely manipulated by the left manipulator 4L, or remotely manipulated by both the right manipulator 4R and the left manipulator 4L.

By using such a surgery assisting system S, it is possible to perform a surgical operation by remotely manipulating one or more surgical instruments, thereby making it possible to shorten the surgical time and also making it possible to perform advanced surgical operations using different types of surgical instruments.

### 2. Linkage between Manipulator and Surgical Instruments

In the description that follows, the term “manipulator 4” will be used to describe features that apply equally to both the right manipulator 4R and the left manipulator 4L, and the terms “right manipulator 4R” and “left manipulator 4L” will be used to describe features unique to the right and left manipulators, respectively. The linkage between the manipulator 4 of the master-side device 1 and the surgical instrument 24 of the slave-side device 2 will be described with reference to the attached drawings.

A configuration of the manipulator 4 of the master-side device 1 will be described first.

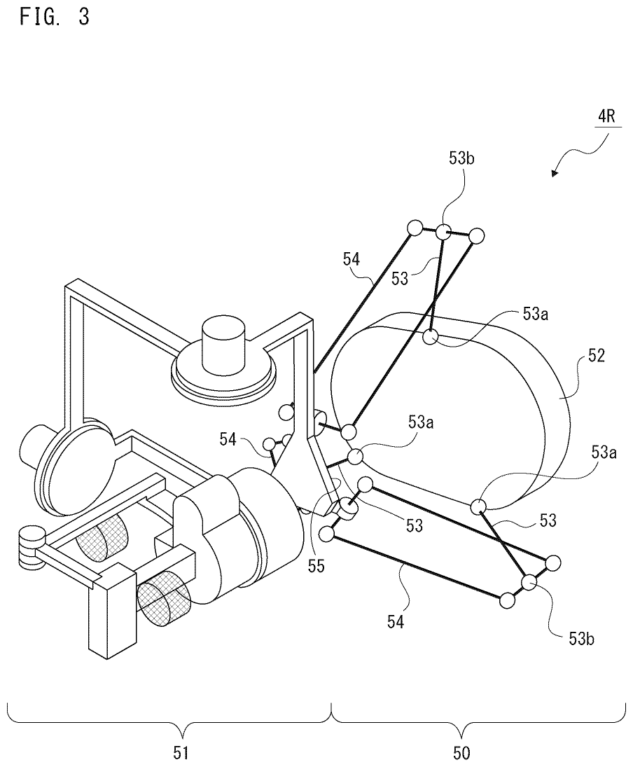

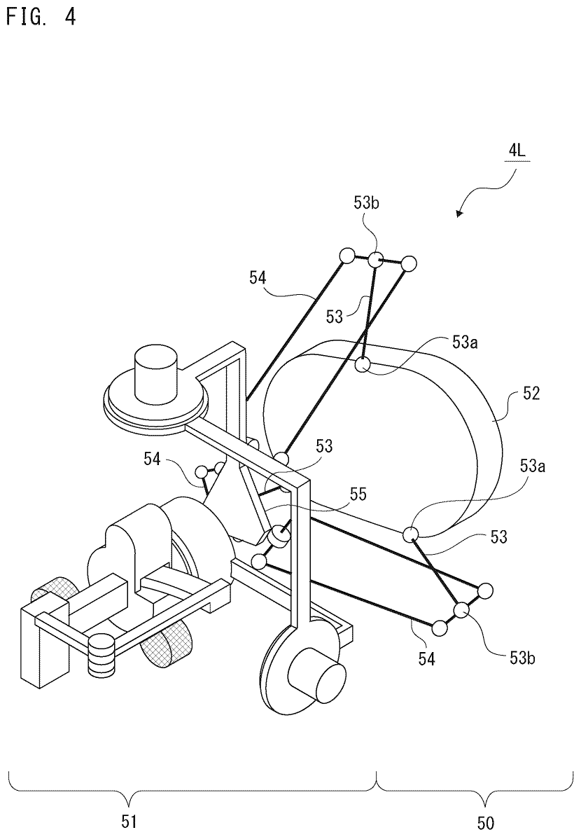

FIG. 3 is a perspective view of the right manipulator 4R, and FIG. 4 is a perspective view of the left manipulator 4L.

The manipulator 4 includes a delta structure 50 for moving the position of the surgical instrument 24 and an attitude changing structure 51 for changing the attitude of the surgical instrument 24.

The right manipulator 4R has a structure in which the left manipulator 4L is flipped horizontally. Except for being horizontally flipped, the structure and function of the right manipulator 4R is substantially the same as the structure and function of the left manipulator 4L. Therefore, for convenience of description, in the following description, the left manipulator 4L will be mainly described by way of example.

The delta structure 50 has a first end attached to the front face part 3a of the base part 3, and a second end serving as a support part that supports the attitude changing structure 51 (best seen in FIG. 1).

The delta structure 50 is configured to position the corresponding surgical instrument 24 in the up and down, left and right, and front and back directions.

Note that in the following description, when describing the direction of the surgical instrument 24, the direction when viewed in the longitudinal direction (direction in which the axis extends) of the surgical instrument 24 will be described by way of example. Specifically, the front and back direction of the surgical instrument 24 refers to the longitudinal direction of the surgical instrument 24, the up and down direction of the surgical instrument 24 refer to the vertical direction in the reference attitude of the surgical instrument 24, and the left and right direction of the surgical instrument 24 refers to a direction perpendicular to the front and back direction and the vertical direction in the reference attitude of the surgical instrument 24.

Therefore, when the attitude of the surgical instrument 24 is rotated by 180 degrees around the axis extending in the longitudinal direction with respect to the reference attitude, that is, when the attitude of the surgical instrument 24 is upside down, the up and down directions of the surgical instrument 24 are reversed.

The surgeon grasps a part of the attitude changing structure 51 supported by the support part of the delta structure 50 and moves the entire attitude changing structure 51 in the up and down, left and right, and front and back directions, thereby turning each part of the delta structure 50 to position the surgical instrument 24. In some embodiments, the part may be a predetermined part.

Specifically, as illustrated in FIGS. 3, 4, 5, and others, the delta structure 50 includes an attaching base part 52 that is attached to the front face part 3a of the base part 3, three joint parts 53 having one ends attached to the attaching base part 52 at slightly above the center, slightly lower right of the center, and slightly lower left of the center, respectively, link structures 54 attached to the other ends of the joint parts 53, and a support part 55 supported by the three link structures 54 by being attached to the other ends of the link structures 54 (see FIGS. 3 and 4).

Each joint part 53 has an end part attached to the attaching base part 52 as an end part 53a, and an end part 53b attached to the corresponding link structure 54.

The spatial position of the support part 55 corresponds to the position of the surgical instrument 24. In other words, the surgeon can remotely move the position of the surgical instrument 24 by moving the position of the support part 55.

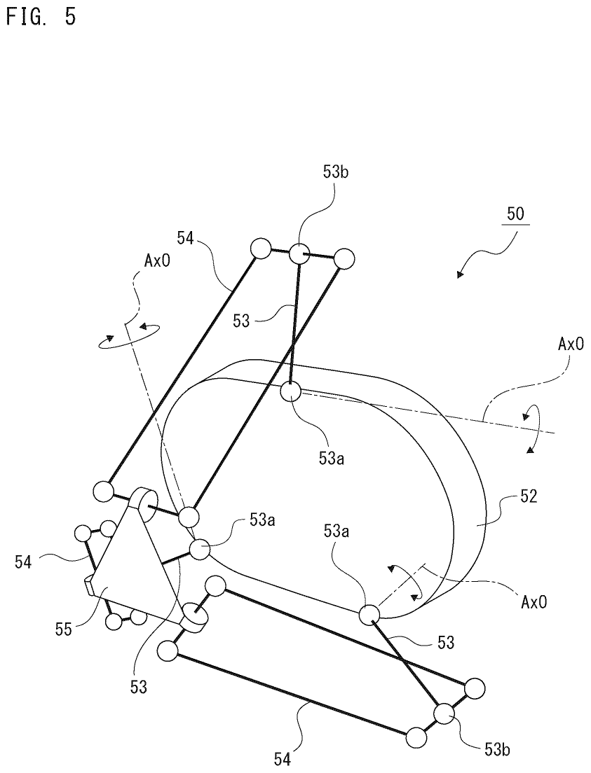

For example, the end part 53a of the joint part 53 is attached to the attaching base part 52 by a ball joint mechanism, and the end part 53a of the joint part 53 is turnable in a direction around a turning axis Ax0 (see FIG. 5). The respective turning axes Ax0 of the end parts 53a of the three joint parts 53 are parallel to the front face part 3a of the base part 3 and are in different directions by 120 degrees.

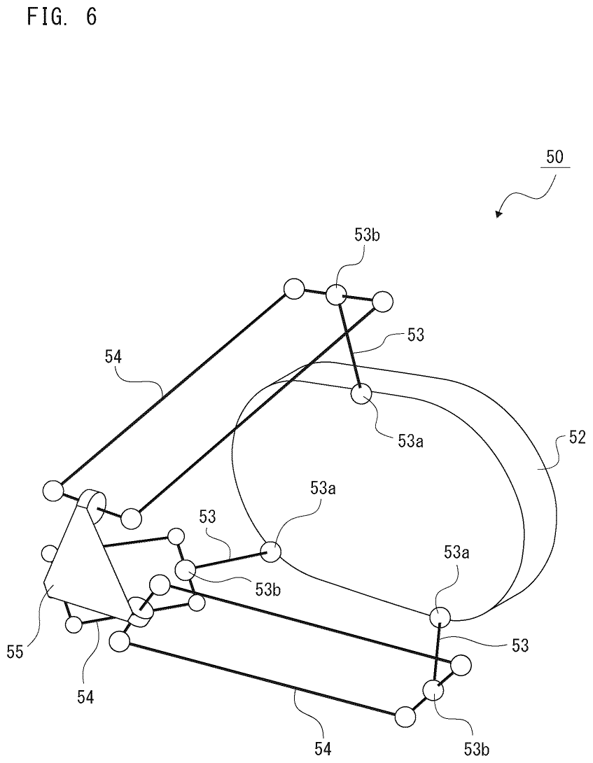

By turning the end part 53a of the joint part 53 in the direction around the turning axis Ax0, the approximate position of the support part 55 in the front and back direction is determined (see FIGS. 5 and 6).

The link structure 54, one end of which is connected to the end part 53b of the joint part 53, includes two parallel links and is deformable into a parallelogram (including a rectangle). In some embodiments, the links may comprise, for example, bars, rods or shafts. However, embodiments are not limited thereto.

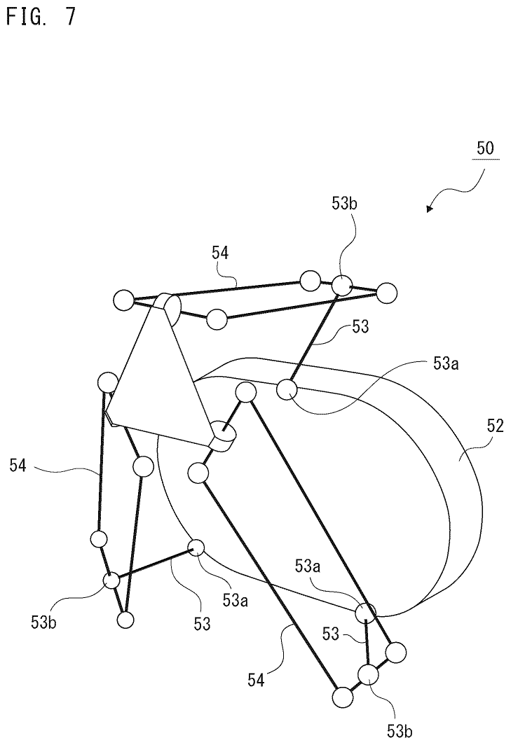

The approximate position of the support part 55 in the up and down and left and right directions is determined by the deformation of the link structure 54. For example, by performing a manipulation to move the entire attitude changing structure 51 upward from the state illustrated in FIG. 5, the link structure 54 is deformed from a substantially rectangle to a parallelogram as illustrated in FIG. 7.

Note that strictly speaking, when the position of the support part 55 in the front and back direction is changed, the angle formed by each joint part 53 and the corresponding link structure 54 changes, and the link structure 54 is also deformed.

Further, when the support part 55 is moved in the up and down direction or the left and right direction, the link structure 54 is deformed, and the end parts 53b of the respective joint parts 53 turn, so that the angle formed by the joint part 53 and the link structure 54 changes.

The support part 55 can be translated in the up and down, right and left, front and back directions in space while maintaining the same attitude (orientation) by the movement of the delta structure 50.

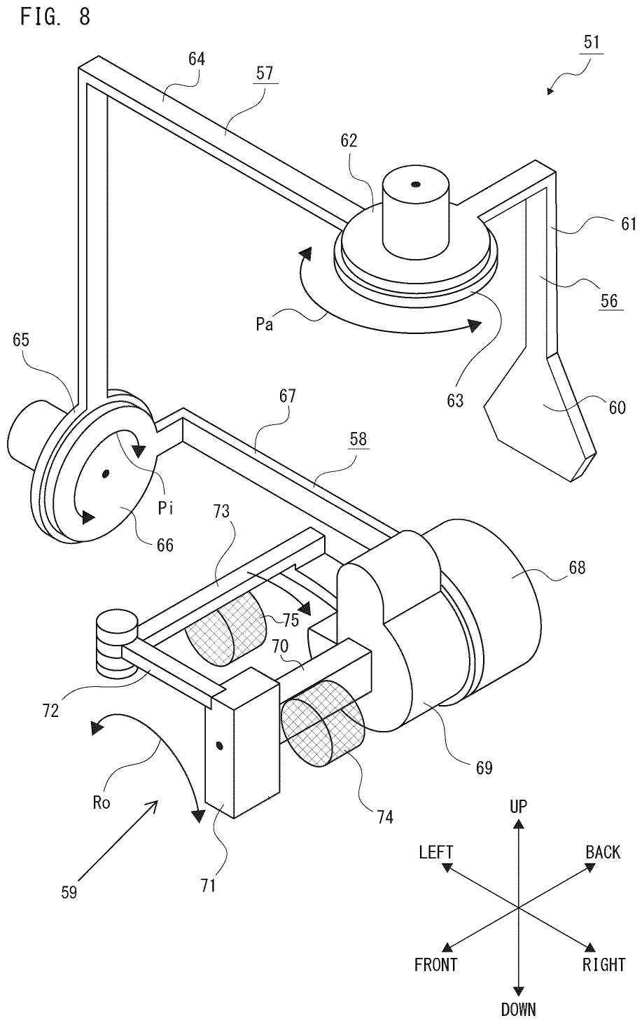

A specific configuration of the attitude changing structure 51 supported by the delta structure 50 by being attached to the support part 55 of the delta structure 50 will be described. Note that FIG. 8 illustrates a schematic configuration of the attitude changing structure 51.

The attitude changing structure 51 includes a first member 56, a second member 57 that is coupled to the first member 56, a third member 58 that is coupled to the second member 57, and a grasping structure 59 that is coupled to the third member 58. In some embodiments, the first to third members 56-58 may comprise plates, bars, rods, discs and/or shafts. However, embodiments are not limited thereto.

The first member 56 includes a supported part 60 that is supported by the support part 55 and is formed in a plate shape facing front and back, a first connecting part 61 that extends from one end part of the supported part 60, and a first turning support part 62 that is continuous with the other end part of the first connecting part 61 and is formed in a disc shape facing up and down.

The second member 57 includes a first turning supported part 63 that is attached to the bottom face part of the first turning support part 62, a second connecting part 64 that is formed in an L-shape extending from the first turning supported part 63 in one of the left and right directions and extending downward from approximately the center part, and a second turning support part 65 that is continuous with the second connecting part 64 and is formed in a disc shape facing left and right.

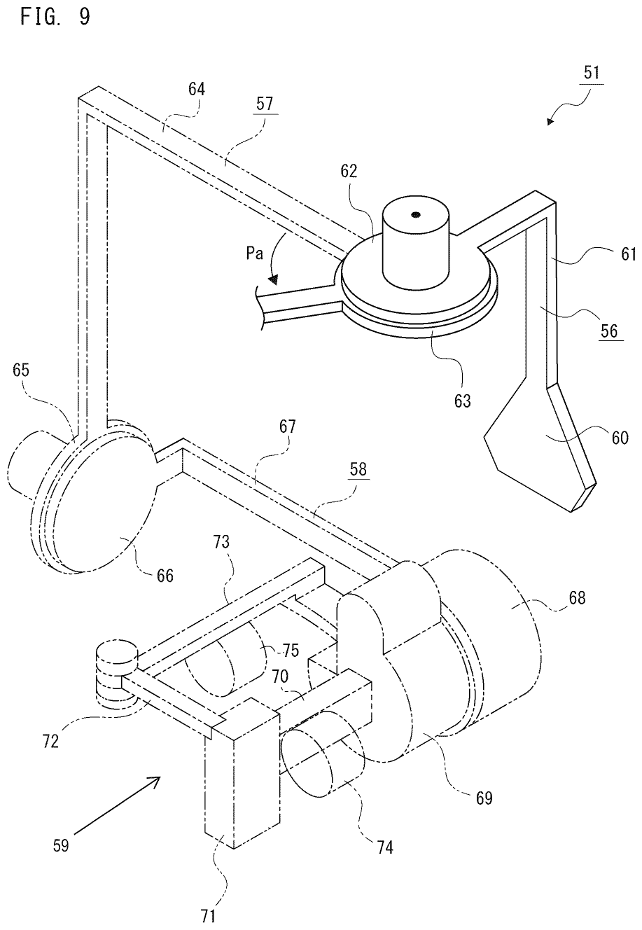

The first turning supported part 63 of the second member 57 is turnable in a direction around an axis extending in the up and down direction with respect to the first turning support part 62 of the first member 56 (see FIGS. 8 and 9). This configuration allows the distal end part of the surgical instrument 24L provided on the slave-side device 2 in correspondence with the left manipulator 4L to swing left and right. This manipulation is referred to as a pan direction turning manipulation Pa. Note that the left and right direction of the distal end part here refers to the left and right direction when the surgical instrument 24L is in the reference attitude, as described above, and depending on the attitude of the surgical instrument 24L, the left and right direction of the distal end part may also be the vertical direction or an oblique direction.

The third member 58 includes a second turning supported part 66 that is attached to the right side face part of the second turning support part 65 and is formed in a disc shape facing left and right, a third connecting part 67 that extends from one end of the second turning supported part 66 to one of the left and right sides, and an attached end part 68 that is continuous with the other end of the third connecting part 67 and is formed in a disc shape facing in the front and back directions and to which the grasping structure 59 is attached.

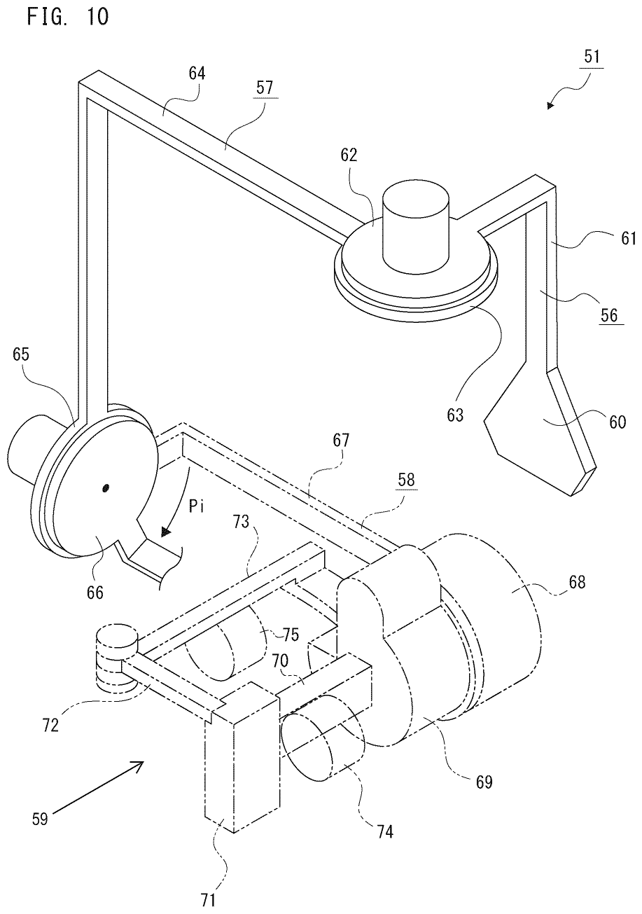

The second turning supported part 66 of the third member 58 is turnable in a direction around an axis extending in the left and right direction with respect to the second turning support part 65 of the second member 57 (see FIGS. 8 and 10). This configuration allows the distal end part of the surgical instrument 24L to swing up and down. This manipulation is referred to as a pitch direction turning manipulation Pi.

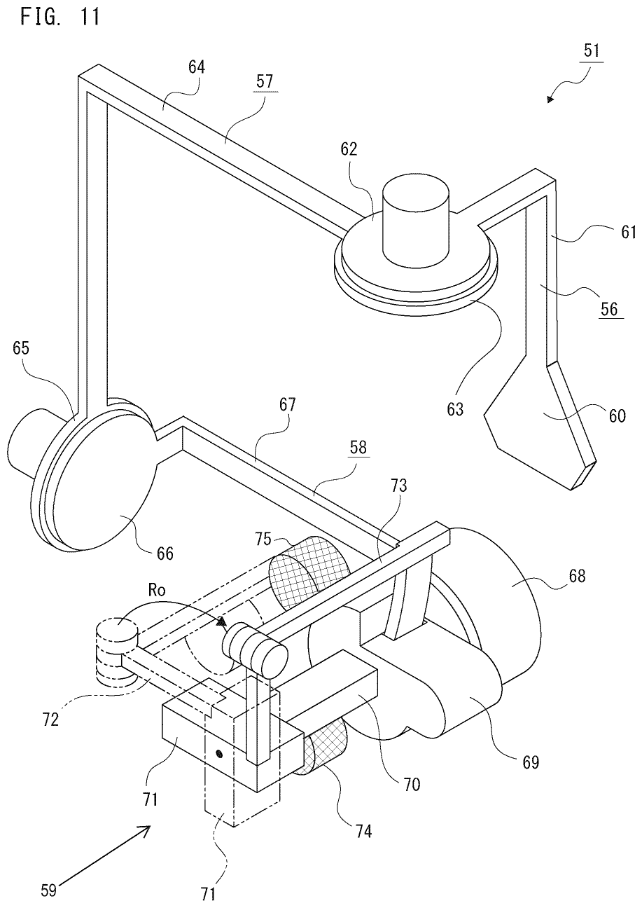

The grasping structure 59 includes an attaching end part 69 that is attached to the attached end part 68 and is formed in a substantially disc shape, a first coupling part 70 that has one end coupled to the front face of the attaching end part 69, a grasping part 71 that is attached to the other end part of the first coupling part 70, a second coupling part 72 that extends laterally from the grasping part 71, and an opening/closing part 73 that is coupled to the end part of the second coupling part 72 opposite to the grasping part 71 and is formed in a substantially L-shape extending forward. In some embodiments, the grasping part 71 may be rectangular with a long side in either the horizontal direction or vertical direction, as illustrated in FIG. 11.

The attaching end part 69 of the grasping structure 59 is turnable in a direction around an axis extending in the front- and back direction with respect to the attached end part 68 of the third member 58 (see FIGS. 8 and 11). This configuration allows the surgical instrument 24L to turn in a direction around the longitudinal axis of the surgical instrument 24L. This manipulation is referred to as a roll direction turning manipulation Ro.

Note that the directions of the attitude changing structure 51 illustrated for the explanation of the pan direction turning manipulation Pa, the pitch direction turning manipulation Pi, and the roll direction turning manipulation Ro are only those in which the attitude changing structure 51 is in the reference attitude illustrated in FIG. 8. Therefore, this does not apply when the attitude of the attitude changing structure 51 becomes a different attitude from the attitude illustrated in FIG. 8 due to turning of each part. For example, when the first turning supported part 63 of the second member 57 is turned by 90 degrees counterclockwise when viewed from above with respect to the first turning support part 62 of the first member 56, the turning axis of the attaching end part 69 of the grasping structure 59 relative to the attaching end part 68 of the third member 58 is an axis extending in the left and right direction rather than in the front and back direction.

The grasping structure 59 includes a first grasping aid 74 that is attached to the side face of the first coupling part 70 and into which a finger of the surgeon is inserted, and a second grasping aid 75 that is attached to the bottom face of the opening/closing part 73 and into which another finger of the surgeon is inserted. For example, in an embodiment, the first grasping aid 74 and the second grasping aid 75 may be loops. However, embodiments are not limited thereto.

For example, the surgeon can manipulate the attitude changing structure 51 suitably by inserting the thumb of the left hand into the first grasping aid 74 and inserting the index finger of the left hand into the second grasping aid 75 while grasping the grasping part 71 with the palm of the left hand.

Further, by moving the fingertips of the thumb and index finger of the left hand closer to each other, the distal end part of the opening/closing part 73 is manipulated to approach the attaching end part 69 (e.g., a closing manipulation Cl). Then, by moving the fingertips of the thumb and index finger of the left hand away from each other, the distal end part of the opening/closing part 73 is manipulated to move away from the attaching end part 69 (e.g., an opening manipulation Op).

By performing the opening manipulation Op and the closing manipulation Cl, the distal end of the forceps as the surgical instrument 24L provided correspondingly in the slave-side device 2 can be closed or opened.

In this way, the attitude changing structure 51 is configured to have four degrees of freedom, and this configuration allows the pan direction turning manipulation Pa to swing the distal end part of the surgical instrument 24 in the pan direction, the pitch direction turning manipulation Pi to swing the distal end part in the pitch direction, the roll direction turning manipulation Ro to turn the distal end part in the roll direction, and an opening/closing manipulation OC to open and close the distal end part of the forceps or the like.

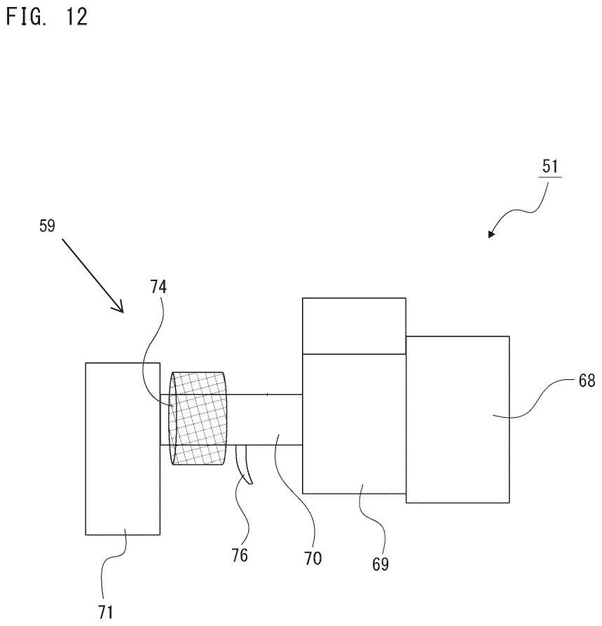

In some embodiments, the grasping structure 59 may include a clutch 76. As illustrated in FIG. 12, the clutch 76 may be attached to the bottom face part of the first coupling part 70 of the grasping structure 59 when the attitude changing structure 51 is in the reference attitude, and may be, for example, a trigger.

The clutch 76 is provided to remove the linkage between the manipulators 4 of the master-side device 1 and the arms 22 and surgical instruments 24 of the slave-side device 2.

Specifically, when the clutch 76 is pulled forward (in the direction away from the attaching end part 69, that is, toward the surgeon), even when a manipulation is performed on the manipulator(s) 4, each part/unit of the slave-side device 2 is disabled.

A specific purpose of manipulating the clutch 76 is to remove the linkage between the master-side device 1 and the slave-side device 2 by the surgeon pulling the clutch 76 toward the surgeon, for example, when any manipulation is difficult because the hand of the surgeon who is grasping the grasping part 71 of the grasping structure 59 moves away from the body. Then, while the clutch 76 is pulled toward the surgeon, the grasping part 71 is translated so as to move the entire attitude changing structure 51, and then the manipulation on the clutch 76 is canceled. As a result, the linkage between the master-side device 1 and the slave-side device 2 is resumed, and the surgeon can resume the surgery with the hand moved to a position where the surgeon is easy to manipulate the manipulator 4.

As illustrated in FIG. 12, the surgeon grasps the grasping part 71 with the palm of the right hand with the thumb of the right hand inserted into the first grasping aid 74 and the index finger inserted into the second grasping aid 75, and in that state, the surgeon can pull the clutch 76 toward the surgeon (the surgeon side) with the middle finger or another finger. Thus, the clutch 76 can be manipulated without changing the position of the hand that can translate or turn the distal end part of the surgical instrument 24. This improves operability.

Note that in some embodiments, the position of the grasping structure 59 of the master-side device 1 is freely changed while the clutch 76 is being manipulated with the linkage between the master-side device 1 and the slave-side device 2 removed, but brakes are applied to each part to disable the pan direction turning manipulation Pa, the pitch direction turning manipulation Pi, and the roll direction turning manipulation Ro, that is, to prevent the positional relationship between the first member 56, the second member 57, the third member 58, and the grasping structure 59 from changing.

In order to perform the roll direction turning manipulation Ro while the clutch 76 is being manipulated, a roll clutch function described later may be used. Note that the surgery assisting system S may include a clutch function for the pan direction turning manipulation Pa and the pitch direction turning manipulation Pi.

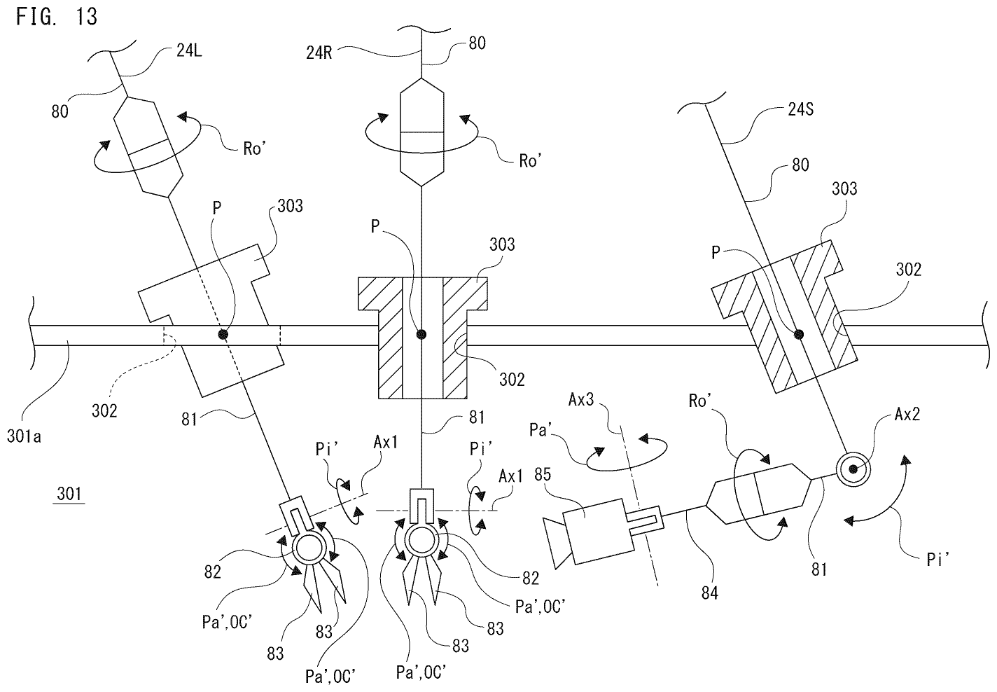

A specific mechanism of the surgical instrument 24 whose attitude changes according to the attitude changing structure 51 is schematically illustrated in FIG. 13.

FIG. 13 illustrates as the surgical instruments 24 the surgical instruments 24R, 24L, and the endoscope 24S.

In an embodiment, the surgical instruments 24R and 24L may be shafts as a whole, for example, and may be configured to include a first shaft part 80 that is held by the surgical instrument holding device 23, a second shaft part 81 that has one end part connected to the distal end part of the first shaft part 80, a turning coupling part 82 that is held at the other end part of the second shaft part 81, and two distal end pieces 83 that are supported by the turning coupling part 82.

The second shaft part 81 is turnable in a direction around the axis of the second shaft part 81 with respect to the first shaft part 80, and turns in response to the roll direction turning manipulation Ro on the grasping structure 59 (turning motion Ro′).

The turning coupling part 82 is turnable in a direction around a first axis Ax1 illustrated in FIG. 13 with respect to the second shaft part 81, and turns in response to the pitch direction turning manipulation Pi on the grasping structure 59 (turning motion Pi′).

Each of the two distal end pieces 83 is turnable with respect to the turning coupling part 82 in a direction around an axis perpendicular to each of the turning axis of the turning motion Ro′ and the turning axis of the turning motion Pi′.

In response to the pan direction turning manipulation Pa on the grasping structure 59, the two distal end pieces 83 turn in the same direction (e.g., a turning motion Pa′). In response to the opening manipulation Op of the opening/closing manipulation OC on the grasping structure 59, the two distal end pieces 83 turn in a direction away from each other (e.g., an opening motion Op′). Furthermore, in response to the closing manipulation Cl of the opening/closing manipulation OC on the grasping structure 59, the two distal end pieces 83 turn in a direction toward each other (e.g., a closing motion Cl′).

In this way, the turning motion Pa′ and an opening/closing motion OC′ of the distal end pieces 83 are achieved.

Next, a configuration example of the endoscope 24S will be described.

The endoscope 24S may comprise a shaft as a whole, for example, and may be configured to include a first shaft part 80 that is held by the surgical instrument holding device 23, a second shaft part 81 that has one end part connected to the distal end part of the first shaft part 80, a third shaft part 84 that is connected to the other end part of the second shaft part 81, and a camera head 85 that is held by the third shaft part 84.

In the above-described example, a configuration has been described in which the position and attitude of the surgical instrument 24R change in response to a manipulation on the right manipulator 4R, and the position and attitude of the surgical instrument 24L change in response to a manipulation on the left manipulator 4L.

However, during surgery, there are situations in which it is advantageous to change the viewing angle of the endoscope 24S. In that case, the position and attitude of the endoscope 24S may be changed according to a manipulation on either the right manipulator 4R or the left manipulator 4L. For example, the linkage between the right manipulator 4R and the endoscope 24S may be started after the linkage between the right manipulator 4R and the surgical instrument 24R is removed.

This operation makes it possible to obtain an image based on an appropriate viewing angle from the endoscope 24S, making it easier to perform surgery.

Specifically, the second shaft part 81 of the endoscope 24S is turnable in a direction around a second axis Ax2 illustrated in FIG. 13 with respect to the first shaft part 80, and turns in response to the pitch direction turning manipulation Pi on the grasping structure 59 (turning motion Pi′).

The third shaft part 84 of the endoscope 24S is turnable in a direction around the axis of the third shaft part 84 with respect to the second shaft part 81, and turns in response to the roll direction turning manipulation Ro on the grasping structure 59 (turning motion Ro′).

The camera head 85 is turnable in a direction around a third axis Ax3 illustrated in FIG. 13 with respect to the third shaft part 84, and turns in response to the pan direction turning manipulation Pa on the grasping structure 59 (turning motion Pa′).

In this way, the change of the attitude of the endoscope 24S is achieved.

Note that to insert the distal end part of the surgical instrument 24 into the body cavity 301, a trocar 303 placed in the corresponding port 302 formed in the abdominal wall 301a is used. To change the position and attitude of the surgical instrument 24, a pivot point P is set inside the trocar 303, that is, near the center of the port 302.

When the distal end part of the surgical instrument 24 is inserted into the body cavity 301 of the patient 300, the position of the surgical instrument 24 is controlled so that a part of the surgical instrument 24 always passes through the pivot point P, preventing a load on the tissue near the body surface of the patient 300 from occurring before and after the change of the position and attitude of the surgical instrument 24 and thus making it possible to ensure safety.

### 3. Functional Configuration

The functional configuration of the surgery assisting system S to smoothly perform surgery will be described.

### <3-1. Functional Configuration of Master-Side Device>

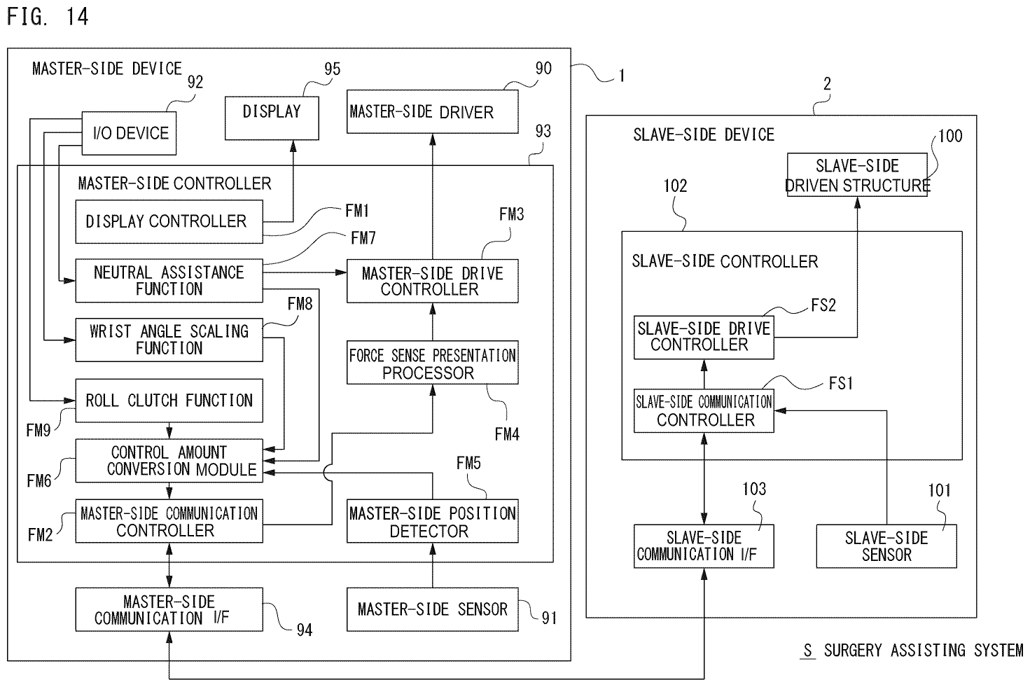

The functional configuration of the master-side device 1 will be described first with reference to FIG. 14. This figure is a functional block diagram of the master-side device 1 and the slave-side device 2 in the surgery assisting system S.

The master-side device 1 includes a master-side driver 90, a master-side sensor 91, an input/output (I/O) device 92, a master-side controller 93, a master-side communication interface (I/F) 94, and a display 95.

The master-side driver 90 is provided as each part of the delta structure 50 and each part of the attitude changing structure 51 in the manipulator 4. Specifically, the master-side driver 90 is an actuator for turning the end part 53a of the joint part 53 in a direction around the turning axis Ax0 illustrated in FIG. 5, an actuator to control a relative turning state between the link structure 54 and the support part 55, or an actuator for turning the second member 57 in the direction of the pan direction turning manipulation Pa with respect to the first member 56 illustrated in FIG. 8.

The master-side driver 90 is driven based on a control signal supplied from the master-side controller 93 to change the position and attitude of each unit of the master-side device 1.

The master-side sensor 91 is provided as a sensor that senses the positional relationship of the parts, and detects, for example, a turning angle representing the turning state of the end part 53a of the joint part 53 in the direction around the turning axis Ax0 illustrated in FIG. 5, a turning angle representing the turning state of the second member 57 with respect to the first member 56 illustrated in FIG. 8, and the like to output the resulting detection signal.

The input/output (I/O) device 92 may include, for example, the clutch 76, the first foot button 5, and/or the second foot button 6, etc. described above. If the first monitor 7, the second monitor 8, and the like each have a touch panel portion, a touch panel portion may also be included in the input/output (I/O) device 92. Note that if a mouse or the like for performing an operation on a menu screen or the like displayed on the second monitor 8 or the like is connected, the mouse or the like may also be included in the input/output (I/O) device 92.

In some embodiments, if the master-side device 1 supports voice input, a microphone or the like included in the master-side device 1 may be included in the input/output (I/O) device 92.

The display 95 may include the first monitor 7 and/or the second monitor 8, etc. described above.

In an embodiment, the master-side controller 93 may be implemented as master-side processing circuitry. The master-side processing circuitry may include a central processing unit (CPU), a read only memory (ROM), and/or a random access memory (RAM), etc. The master-side controller 93 may implement one or more of the functions described above. For example, a memory such as the ROM may store one or more programs or the RAM may be loaded with one or more programs. The CPU may access the memory and execute at least one of the one or more programs to cause the CPU to implement one or more of the functions described above.

Specifically, the master-side controller 93 includes a display control controller FM1, a master-side communication controller FM2, a master-side drive controller FM3, a force sense presentation processor FM4, a master-side position detector FM5, a control amount conversion module FM6, a neutral assistance function FM7, a wrist angle scaling function FM8, and a roll clutch function FM9. In some embodiments, the master-side controller 93 may implement one or more of the display control controller FM1, the master-side communication controller FM2, the master-side drive controller FM3, the force sense presentation processor FM4, the master-side position detector FM5, the control amount conversion module FM6, the neutral assistance function FM7, the wrist angle scaling function FM8, and the roll clutch function FM9.

The display control controller FM1 performs display control on the first monitor 7 and the second monitor 8. In the an embodiment, the display control controller FM1 causes the second monitor 8 to display setting screens for various settings. On each setting screen, a drop-down list, buttons, and the like are arranged on the input/output (I/O) device 92, and manipulation information for the input/output (I/O) device 92 is notified to the neutral assistance function FM7, the wrist angle scaling function FM8, and the roll clutch function FM9.

The display control controller FM1 receives image data captured by the endoscope 24S serving as the surgical instrument 24 included in the slave-side device 2 through the master-side communication interface (I/F) 94, and outputs the captured image to the first monitor 7.

The master-side communication controller FM2 performs processing for transmitting and receiving various types of information to and from the slave-side device 2 using the master-side communication interface (I/F) 94.

The master-side drive controller FM3 controls various actuators each provided as the master-side driver 90 in order to control each part/unit of the master-side device 1.

Note that the master-side drive controller FM3 may be controlled by other functions or parts/units in order to implement various functions of the master-side controller 93. In other words, the master-side drive controller FM3 is capable of executing processing for controlling the master-side driver 90 based on commands output from other functions or parts/units.

One example is force sense presentation control. Specifically, the force sense presentation processor FM4 performs processing for presenting to the surgeon a force sense as if the surgeon were handling the surgical instrument 24 with the surgeon's hand, in accordance with the movement of the surgical instrument 24 such as forceps included in the slave-side device 2. For example, when the surgical instrument 24 hits an obstacle, the master-side driver 90 is driven in a direction in which the manipulation input by the surgeon is canceled, making the force necessary to move the surgical instrument 24 larger than usual, so that the surgeon can sense that the surgical instrument 24 hits the obstacle.

In order to perform such a control, the force sense presentation processor FM4 instructs the master-side drive controller FM3.

The master-side controller 93 performs a control to drive each part/unit of the slave-side device 2 in accordance with a surgeon's manipulation on the master-side device 1.

Specifically, the master-side position detector FM5 detects the position, attitude, and the like of the master-side driver 90 based on the output signal from the master-side sensor 91, and outputs the result of detection to the control amount conversion module FM6.

The control amount conversion module FM6 calculates the amount of change in the master-side driver 90 from the position, attitude, and the like of the master-side driver 90 input from the master-side position detector FM5. The control amount conversion module FM6 converts the amount of change into an amount of control for each drive unit of the slave-side device 2 based on the amount of change. Note that the position and attitude of each surgical instrument 24 of the slave-side device 2 are changed so that the surgical instrument 24 passes through the pivot point P set near the corresponding port 302 of the patient 300. The control amount conversion module FM6 performs processing of converting the translational movement of the support part 55 of the master-side device 1 into a turning motion or other type of motion of the drive unit of the slave-side device 2 so that the surgical instrument 24 passes through the pivot point P.

The amount of control (amount of drive) calculated for each driver of the slave-side device 2 by the control amount conversion module FM6 is transmitted from the master-side communication interface (I/F) 94 to the slave-side device 2 through processing performed by the master-side communication controller FM2.

The neutral assistance function FM7, the wrist angle scaling function FM8, and the roll clutch function FM9 can be regarded as auxiliary functions for performing surgery smoothly.

### <3-1-1. Neutral Assistance Function>

The neutral assistance function FM7 is a function for guiding the delta structure 50 of the manipulator 4 included in the master-side device 1 to an appropriate position.

For example, the neutral assistance function FM7 is a function that is started in response to simultaneous manipulation (manipulation of pulling toward the surgeon) of the clutch 76 provided in the attitude changing structure 51 of the left manipulator 4L and the clutch 76 provided in the attitude changing structure 51 of the right manipulator 4R. Note that this way of manipulation is just an example, and the function may be executed by voice operation, or, for example, the function may be executed by pressing the first foot button 5 and the second foot button 6 at the same time. In some embodiments, the function may be executed by inputting a manipulation pattern to the clutch 76 or the like. In some embodiment, the manipulation pattern may be predetermined.

The neutral assistance function FM7 is a function that moves only the delta structure 50 of the master-side device 1 to a position without moving each part/unit of the slave-side device 2. In some embodiments, the position may be predetermined. Therefore, to execute the neutral assistance function FM7, the control amount conversion module FM6 is notified that the linkage with the slave-side device 2 is to be removed (suspended). In response to this notification, the control amount conversion module FM6 calculates as “0” an amount of motion of the turning motion of the driver of the slave-side device 2 and other types of motion, regardless of the result of detection by the master-side sensor 91.

The neutral assistance function FM7 performs processing for guiding the delta structure 50 to an appropriate position in a state where the linkage with the slave-side device 2 is terminated.

Note that while only software may be used to terminate the linkage between the master-side device 1 and the slave-side device 2 as described above, hardware structure may be used.

For example, each movable part of the slave-side device 2 may have an electromagnetic brake as a non-excitation type of brake that is configured to apply a brake and maintain the position and attitude when the power supply is cut off. In this case, the power supply to the electromagnetic brake is stopped to apply a brake to each movable part, thereby maintaining the position and attitude of each movable part. This configuration and operation can be achieved without any modification to the calculation of the amount of motion by the control amount conversion module FM6.

The delta structure 50 of the master-side device 1 has a range of movement in the up and down direction, left and right direction, and front and back direction, which limits the possible range of the position of the attitude changing structure 51 attached to the delta structure 50 (or the position of the grasping structure 59) in each direction. In some embodiments, the range of movement may be predetermined.

For example, if the surgeon performs a manipulation near the end of the range of movement of the grasping structure 59, the range of movement of the delta structure 50 will reach the range limit, making it impossible to perform the intended manipulation, which may fail to provide adequate treatment to the patient 300.

In order to address such a situation, the delta structure 50 may be guided by the neutral assistance function FM7.

Specifically, the neutral assistance function FM7 moves each part of the left and right delta structures 50 so that the left and right grasping structures 59 are each located approximately at the center of the range of movement in each direction.

As a result, the grasping structure 59 grasped by the surgeon can resume the surgery with a certain range of movement remaining on either side in each of the up and down direction, left and right direction, and front and back direction, and thus, adequate treatment can be provided.

For this purpose, the neutral assistance function FM7 instructs the master-side drive controller FM3.

Note that, in order to guide the grasping structure 59 to a position, force sense presentation may be performed to convey the direction in which the palm of the surgeon who is grasping the grasping structure 59 should be moved. In some embodiments, the position may be predetermined.

After the grasping structure 59 is moved to the position by the neutral assistance function FM7, the linkage between the master-side device 1 and the slave-side device 2 can be resumed. The linkage may be resumed automatically, or may be configured not to be resumed unless a manual manipulation is performed.

Note that when the clutch 76 is pulled toward the surgeon, the linkage between the master-side device 1 and the slave-side device 2 is terminated, and when the clutch 76 is released from being pulled toward the surgeon, the linkage between the master-side device 1 and the slave-side device 2 is resumed. However, when the left and right clutches 76 are manipulated simultaneously, even when the left and right clutches 76 are released from being pulled toward the surgeon during automatic control of the grasping structure 59 by the neutral assistance function FM7, it is advantageous from the viewpoint of safety that the linkage between the master-side device 1 and the slave-side device 2 is not resumed unless the automatic control is completed.

Further, when the left and right clutches 76 are released from being pulled toward the surgeon during automatic control of the grasping structure 59 by the neutral assistance function FM7, the automatic control by the neutral assistance function FM7 may be canceled to immediately resume the linkage between the master-side device 1 and the slave-side device 2.

In some embodiments, it is possible to switch on/off of the neutral assistance function FM7. This switching is realized by performing an operation on a setting screen displayed on the second monitor 8.

An example of a setting screen G1 will be described with reference to the attached drawings.

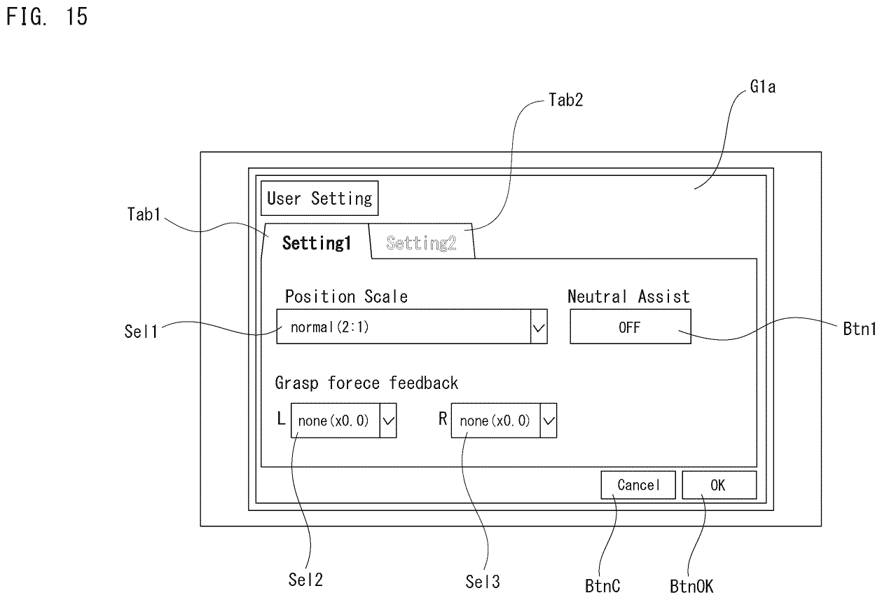

The setting screen G1 is configured as a screen with two tabs, and FIG. 15 illustrates a state in which a first setting tab Tab1 displayed as “Setting1” is selected. This screen is referred to as a setting screen G1a.

The setting screen G1a includes a position scaling selection field Sel1, a neutral assistance button Btn1, a left force sense presentation strength selection field Sel2, a right force sense presentation strength selection field Sel3, a cancel button BtnC, and an execution button BtnOK.

Among these, a control for switching on/off of the neutral assistance function FM7 is the neutral assistance button Btn1.

The current state is displayed on the neutral assistance button Btn1, and on and off are switched by operating the neutral assistance button Btn1.

By operating the cancel button BtnC, the setting screen G1 can be closed without reflecting the settings set on the setting screen G1a.

Further, by operating the execution button BtnOK, the settings screen G1 can be closed with the settings set on the settings screen G1a reflected.

### <3-1-2. Wrist Angle Scaling Function>

The wrist angle scaling function FM8 is a function for changing the amount of turning of the turning motion Pa′ for the pan direction turning manipulation Pa, the amount of turning of the turning motion Pi′ for the pitch direction turning manipulation Pi, and the amount of turning of the turning motion Ro′ for the roll direction turning manipulation Ro.

The possible amount of movement of the human wrist is approximately fixed, and for example, it is difficult to rotate the wrist by 360 degrees while performing precise manipulations. The wrist angle scaling function FM8 is a function to address such a problem.

The ratio between the amount of manipulation and the amount of turning can be achieved by manipulating the input/output (I/O) device 92. The wrist angle scaling function FM8 notifies the control amount conversion module FM6 of the ratio set based on the manipulation of the input/output (I/O) device 92. Based on that notification, the control amount conversion module FM6 obtains the final amount of motion by multiplying the amount of manipulation of each part of the slave-side device 2 calculated from the amount of manipulation by the above-mentioned ratio.

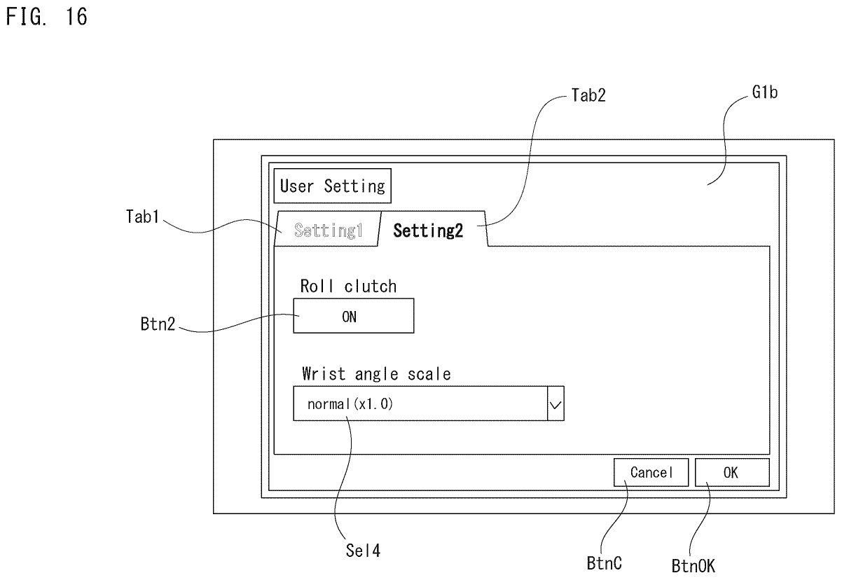

The input/output (I/O) device 92 for determining the ratio between the amount of manipulation and the amount of turning may be a mechanical knob such as a dial knob, or may be a control displayed on a screen realized by software. An example is illustrated in FIG. 16.

FIG. 16 illustrates a state in which a second setting tab Tab2 displayed as “Setting2” is selected on the setting screen G1 with two tabs. This screen is referred to as a setting screen G1b.

A roll clutch button Btn2, a ratio selection field Sel4, a cancel button BtnC, and an execution button BtnOK are arranged on the setting screen G1b.

Among these, a control for determining the ratio between the amount of manipulation and the amount of turning in the wrist angle scaling function FM8 is the ratio selection field Sel4.

The ratio selection field Sel4 allows a selection from three options: “1.0”, “1.5”, and “2.0”. For example, for a selection of “2.0”, when the pan direction turning manipulation Pa is performed by 30 degrees in the master-side device 1, the distal end part of the surgical instrument 24 in the slave-side device 2 is controlled to turn by 60 degrees.

Note that the options to be selected in the ratio selection field Sel4 may include a value less than 1.0. This range of ratios allows for performing precise work.

Further, a slider control may be provided instead of the ratio selection field Sel4 so that the ratio can be changed almost operationlessly.

The operations on the cancel button BtnC and the execution button BtnOK are the same as those on the setting screen G1a, and thus, the description thereof will be omitted.

Note that the master-side controller 93 may have a scaling function for adjusting the amount of turning of the turning motion Pa′ for the pan direction turning manipulation Pa, a scaling function for adjusting the amount of turning of the turning motion Pi′ for the pitch direction turning manipulation Pi, and a scaling function for adjusting the amount of turning of the turning motion Ro′ for the roll direction turning manipulation Ro. In addition, these scaling functions may be set with different ratios.

Further, the master-side controller 93 may not have one or some of the scaling functions.

### <3-1-3. Roll Clutch Function>

The roll clutch function FM9 is a function that determines whether or not to perform the turning motion Ro′ of the distal end part of the surgical instrument 24 of the slave-side device 2 in response to the roll direction turning manipulation Ro.

This function aims to address the problem of difficulty in performing surgery due to the range of movement of the human wrist.

For example, when a turning manipulation of 180 degrees is performed as the roll direction turning manipulation Ro, specifically, when the wrist turns so that the palm turns from facing down to facing up, the distal end part of the surgical instrument 24 performs the turning motion Ro′ of 180 degrees accordingly. In this state, if it is desired to perform the turning motion Ro′ in the same direction, it may be difficult due to the range of movement of the surgeon's wrist. In that case, by the roll clutch function FM9, the state is changed to such a state that the turning motion Ro′ of the distal end part of the surgical instrument 24 is not performed even when the roll direction turning manipulation Ro is performed.

Next, the surgeon turns the wrist so that the palm faces down again, and then, by the roll clutch function FM9, the state is changed to such a state that the roll direction turning motion Ro′ of the distal end part of the surgical instrument 24 is performed again.

Repeating this process makes it possible to perform the turning motions Ro′ in the same direction any number of times.

The input/output (I/O) device 92 for turning on/off the linkage between the roll direction turning manipulation Ro and the turning motion Ro′ may be mechanical such as a button, or may be a control displayed on a screen realized by software.

In the example illustrated in FIG. 16, the roll clutch button Btn2 on the setting screen G1b is a control for the roll clutch function FM9.

The current state is displayed on the roll clutch button Btn2, and by operating the roll clutch button Btn2, the linkage between the manipulation in the roll direction and the turning motion can be switched on/off.

### <3-2. Functional Configuration of Slave-side Device>

Returning to FIG. 14, the slave-side device 2 may include a slave-side driven structure 100, a slave-side sensor 101, a slave-side controller 102, and a slave-side communication interface (I/F) 103.

The slave-side driven structure 100 comprises a movable part of the slave-side device 2. The slave-side device 2 allows for the distal end of each surgical instrument 24 to be moved under control of the master-side device 1 in order to perform surgery on a patient. The movable part may include one or more movable parts of the slave-side device 2 that are movable in order to achieve this movement to perform the surgery. In some embodiments, the movable part may include a structure from the stage 21 to a distal end of the surgical instrument 24 as a whole. In some embodiments, the movable part may include each part of the arm 22, each part of the surgical instrument holding device 23 attached to the arm 22, and each part of the surgical instrument 24 attached to the surgical instrument holding device 23, which are included in the slave-side device 2. For example, in some embodiments, the movable part of the slave-side device 2 may include the joints or turning parts of a given arm 22, the gimbal of the surgical instrument holding device 23, and/or the forceps of the surgical instrument 24. However, these are only examples of the movable part and, in some embodiments, the movable part of the given arm 22 may include fewer or more than these parts.

The slave-side driven structure 100 is driven based on a control signal supplied from the slave-side controller 102 to perform various translational motions and turning motions, so that the position and attitude of the distal end part of the surgical instrument 24 can be changed.

The slave-side sensor 101 senses the positional relationship of the movable parts of the slave-side device 2, and outputs a detection signal corresponding to, for example, the turning position of the second shaft part 81 with respect to the first shaft part 80 illustrated in FIG. 13, the turning position of the distal end pieces 83 with respect to the turning coupling part 82, and the like.

In some embodiments, the slave-side sensor 101 may include an image sensor or the like included in the endoscope 24S.

In some embodiments, the slave-side controller 102 may be implemented as slave-side processing circuitry. The slave-side processing circuitry may include a CPU, ROM, and/or RAM, etc. The slave-side controller 102 may implement one or more functions described above. For example, a memory such as the ROM may store one or more programs or the RAM may be loaded with one or more programs. The CPU may access the memory and execute at least one of the one or more programs to cause the CPU to implement one or more of the functions described above.

Specifically, the slave-side controller 102 includes a slave-side communication controller FS1 and a slave-side drive controller FS2. In some embodiments, the slave-side controller 102 may implement one or more of the slave-side communication controller FS1 and the slave-side drive controller FS2.

The slave-side communication controller FS1 performs processing for transmitting and receiving various types of information to and from the master-side device 1 using the slave-side communication interface (I/F) 103.

The slave-side drive controller FS2 controls various actuators each provided as the slave-side driven structure 100 in order to control the drivers provided as parts of the slave-side device 2. Specifically, the slave-side drive controller FS2 outputs a control signal to each actuator of the slave-side device 2 based on the amount of control (amount of drive) calculated by the control amount conversion module FM6 of the master-side controller 93.

### 4. Processing Flow

An example of the flow of processing will be described that is executed by the master-side controller 93 to implement the above-described various functions of the master-side device 1.

### <4-1. Processing Related to Neutral Assistance Function>

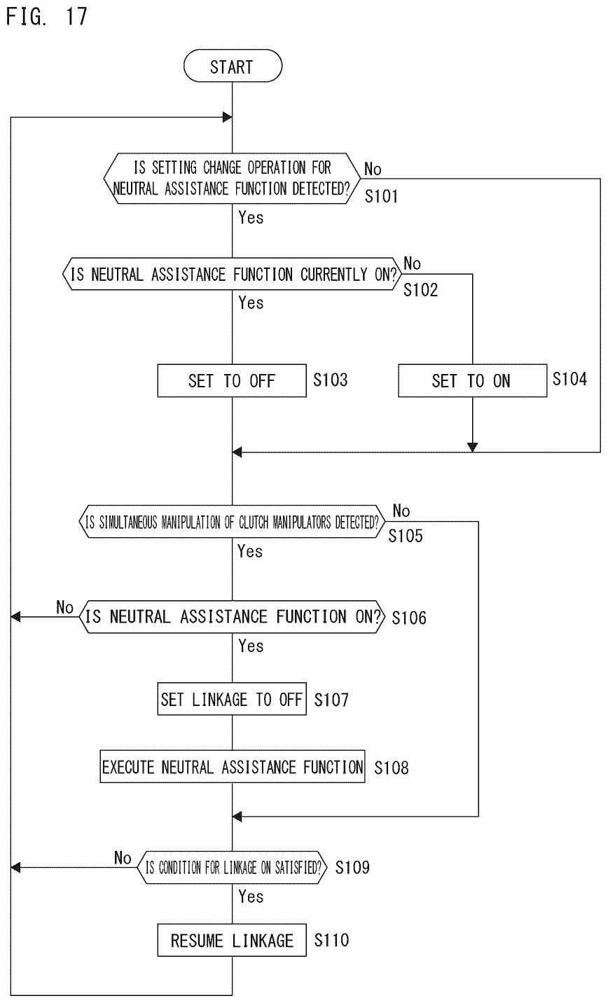

As processing related to the neutral assistance function FM7, the master-side controller 93 executes a series of processes illustrated in FIG. 17.

In operation S101, the master-side controller 93 determines whether or not a setting change operation for the neutral assistance function has been detected. The setting change operation is, for example, an operation of pressing the neutral assistance button Btn1 arranged on the setting screen G1a to change the current state, and then pressing the execution button BtnOK.

If it is determined that no setting change operation has been detected, the master-side controller 93 proceeds to operation S105 without executing processes of operation S102 to operation S104.

On the other hand, if it is determined that a setting change operation has been detected, the master-side controller 93 determines in operation S102 a setting indicating whether or not to start the neutral assistance function in response to a manipulation input such as simultaneously pressing the two clutches 76 is on or off. In some embodiments, the manipulation input may be predetermined.

If the neutral assistance function is on, the master-side controller 93 sets the function to off in operation S103.

On the other hand, if the neutral assistance function is off, the master-side controller 93 sets the function to on in operation S104.

After executing operation S103 or operation S104, or after making a “No” determination in operation S101, the master-side controller 93 determines in operation S105 whether or not the manipulation input, that is, the simultaneous manipulation input of the two clutches 76 has been detected.

If it is determined that the manipulation input has not been detected, the master-side controller 93 proceeds to operation S109.

On the other hand, if it is determined that the manipulation input has been detected, the master-side controller 93 determines in operation S106 whether or not the neutral assistance function is on.

If it is determined that the neutral assistance function is on, the master-side controller 93 removes (suspends) the linkage between the master-side device 1 and the slave-side device 2 in operation S107, and executes the neutral assistance function in operation S108.

Specifically, the master-side controller 93 moves each part of the left and right delta structures 50 so that the left and right grasping structures 59 are each located approximately at the center of the range of movement in each direction.

Note that the neutral assistance function may be executed using the force sense presentation by the force sense presentation processor FM4, as described above.

On the other hand, if it is determined that the neutral assistance function is off, the master-side controller 93 returns to the process of operation S101.

After executing operation S108, the master-side controller 93 determines in operation S109 whether or not a condition for resuming the linkage between the master-side device 1 and the slave-side device 2 is satisfied.

If it is determined that the condition for resuming the linkage is satisfied, the master-side controller 93 resumes the linkage between the master-side device 1 and the slave-side device 2 in operation S110, and returns to the process of operation S101.

On the other hand, if it is determined that the condition for resuming the linkage is not satisfied, the processing returns to operation S101 with the linkage between the master-side device 1 and slave-side device 2 being suspended.

Repeatedly executing the series of processes illustrated in FIG. 17 at time intervals during the surgery assisting system S running allows the surgeon to start the neutral assistance function at any timing. Further, the series of processes illustrated in FIG. 17 ends, for example, when the surgery assisting system S is powered off.

### <4-2. Processing Related to Wrist Angle Scaling Function>

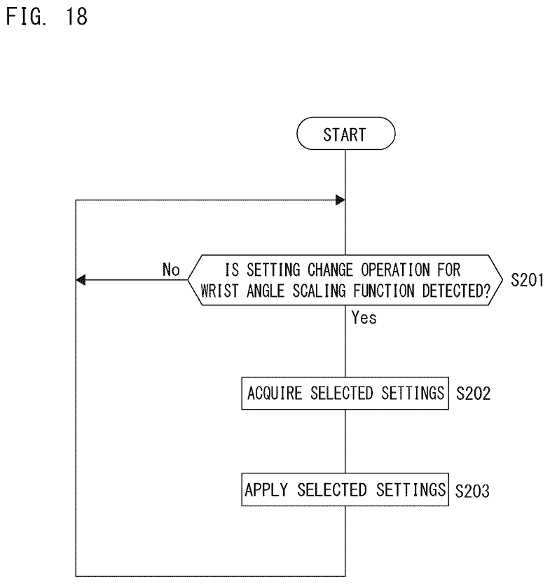

As processing related to the wrist angle scaling function FM8, the master-side controller 93 executes a series of processes illustrated in FIG. 18.

First, the master-side controller 93 determines in operation S201 whether or not a setting change operation for the wrist angle scaling function has been detected. The setting change operation is, for example, an operation of selecting a different ratio from the current ratio from the ratio selection field Sel4 arranged on the setting screen G1b and then pressing the execution button BtnOK.

If it is determined that no setting change operation has been detected, the master-side controller 93 executes the process of operation S201 again.

On the other hand, if it is determined that a setting change operation has been detected, the master-side controller 93 acquires the selected settings in operation S202, and applies the selected settings in operation S203. Applying the selected settings is performed, for example, by notifying the control amount conversion module FM6 of ratio information. The control amount conversion module FM6 obtains the amount of motion of each movable part of the slave-side device 2 based on the notified ratio information.

After ending the process of operation S203, the master-side controller 93 returns to the process of operation S201.

Repeatedly executing the series of processes illustrated in FIG. 18 at time intervals during the surgery assisting system S running allows the surgeon to change the ratio between the amount of manipulation and the amount of motion at any timing based on the wrist angle scaling function. Further, the series of processes illustrated in FIG. 18 ends, for example, when the surgery assisting system S is powered off.

### <4-3. Processing Related to Roll Clutch Function>

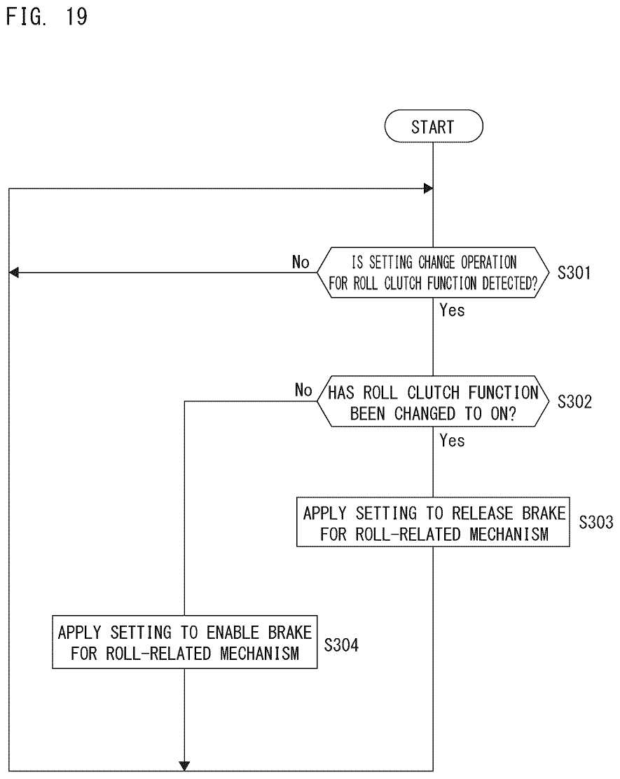

As processing related to the roll clutch function FM9, the master-side controller 93 executes a series of processes illustrated in FIG. 19.

First, in operation S301, the master-side controller 93 determines whether or not a setting change operation for the roll clutch function has been detected. The setting change operation is, for example, an operation of pressing the roll clutch button Btn2 arranged on the setting screen G1b to change the current state, and then pressing the execution button BtnOK.

If it is determined that no setting change operation has been detected, the master-side controller 93 executes the process of operation S301 again.

On the other hand, if it is determined that a setting change operation has been detected, the master-side controller 93 determines in operation S302 whether or not the roll clutch function has been changed from off to on.

If it is determined that the change from off to on has been made, the master-side controller 93 applies a setting for releasing the brake of the roll-related mechanism when the clutch 76 is pulled toward the surgeon in operation S303. This allows the surgeon to perform the roll direction turning manipulation Ro in addition to a translational motion of the grasping structure 59 while pulling the clutch 76 toward the surgeon.

For example, for the two distal end pieces 83 of the surgical instrument 24 as forceps having different shapes, there are cases where it is advantageous to perform the turning motion Ro′ of 180 degrees in order to bring the distal end part of the surgical instrument 24 into a suitable attitude. The roll clutch function is suitable in such cases.

If it is determined in operation S302 that the roll clutch function has been changed from on to off, the master-side controller 93 applies a setting for applying the brake of the roll-related mechanism when the clutch 76 is pulled toward the surgeon in operation S304. As a result, even when the clutch 76 is pulled toward the surgeon, the brake is applied so that the attaching end part 69 does not turn relative to the attached end part 68 illustrated in FIG. 8.

After ending the process of operation S303 or the process of operation S304, the master-side controller 93 returns to the process of operation S301.

Repeatedly executing the series of processes illustrated in FIG. 19 at time intervals during the surgery assisting system S running allows the surgeon to switch the roll clutch function between on and off at any timing. Further, the series of processes illustrated in FIG. 19 ends, for example, when the surgery assisting system S is powered off.

### 5. Modification Example

The surgery assisting system S may be capable of scaling the amount of manipulation and the actual amount of movement to perform translational movement of the distal end part of the surgical instrument 24. For example, in the setting screen G1a illustrated in FIG. 15, an example is illustrated in which the position scaling selection field Sel1 is arranged.

The position scaling selection field Sell allows a selection of ratios between the amount of manipulation for the surgeon to perform a manipulation for translating the grasping structure 59 using the delta structure 50 and the amount of movement for the distal end part of the surgical instrument 24 to translate in accordance with that manipulation.

The position scaling selection field Sell allows a selection from “2:1”, “3:1”, “1:1”, and others. Providing options for reducing the amount of movement relative to the amount of manipulation makes it possible to suitably respond to delicate surgical operations.

By the above-described neutral assistance function FM7, processing has been described that is performed for guiding the grasping structure 59 to approximately the center of the range of movement in the up and down, left and right, and front and back directions in order to facilitate the translational movement of the distal end part of the surgical instrument 24.

In some embodiments, by the neutral assistance function FM7, force sense presentation may be performed to guide the attitude changing structure 51 to approximately the center of each range of movement for the pan direction turning manipulation Pa, the pitch direction turning manipulation Pi, and the roll direction turning manipulation Ro.

In some embodiments, the setting screen G1 may be configured to allow the surgeon to select a target manipulation for which the neutral assistance function is to be executed. For example, a configuration may be provided in which the pan direction turning manipulation Pa and the pitch direction turning manipulation Pi are not applied with the neutral assistance function, while the roll direction turning manipulation Ro is selectable for that function. This configuration allows customization to suit the surgeon's preferences, as well as arrangements suitable for various situations.

In FIG. 16, as the wrist angle scaling function FM8, an example has been described in which the ratio selection field Sel4 is arranged to implement a scaling function for adjusting the amount of turning of the turning motion Pa′ for the pan direction turning manipulation Pa, a scaling function for adjusting the amount of turning of the turning motion Pi′ for the pitch direction turning manipulation Pi, and a scaling function for adjusting the amount of turning of the turning motion Ro′ for the roll direction turning manipulation Ro.

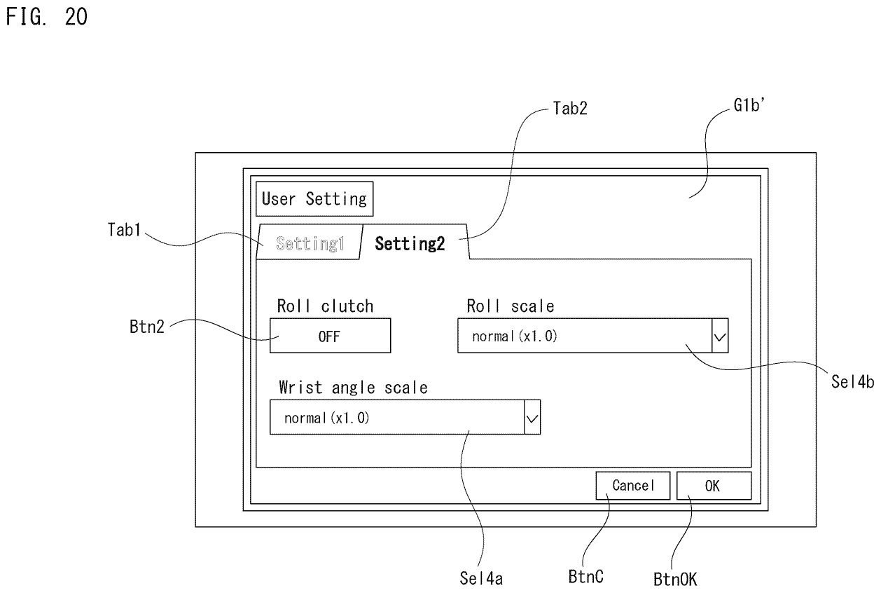

FIG. 20 illustrates an example of the wrist angle scaling function FM8 in which a selection field for scaling in the pan direction and pitch direction and a selection field for scaling in the roll direction are separate.

Specifically, on a setting screen G1b′, a roll clutch button Btn2, a pan and pitch ratio selection field Sel4a, a roll ratio selection field Sel4b, a cancel button BtnC, and an execution button BtnOK are arranged.

It is possible to select different ratios in the pan and pitch ratio selection field Sel4a and the roll ratio selection field Sel4b, respectively. This configuration allows customization to suit the surgeon's preferences, as well as arrangements suitable for various situations.

A surgery assisting device in the surgery assisting system S described above is a master-side device 1 for remotely manipulating a slave-side device 2, and may include: a first manipulator (the grasping part 71 or the grasping structure 59) having a range of movement and used to manipulate a movable part (a movable part of the arm 22, a movable part of the surgical instrument holding device 23, and a movable part of the surgical instrument 24, that is, the slave-side driven structure 100) of the slave-side device; a second manipulator (the clutch 76) provided corresponding to the first manipulator; and a controller (the master-side controller 93) configured to control the movable part in response to a manipulation input to the first manipulator, wherein when a manipulation is performed on the second manipulator, the controller performs processing for moving a position of the first manipulator to a position (approximately the center of the range of movement) within the range of movement.

This processing is executed by the neutral assistance function FM7 described above.

This configuration and operation makes it possible to move, when the first manipulator is located near the end of the range of movement, the first manipulator to a position where it is easy for a surgeon to manipulate, specifically, to approximately the center of the range of movement.

Therefore, it is possible to prevent the surgeon from being able to perform the intended manipulation due to reaching the limit of the range of movement, making it easier to perform surgery. Further, it is possible to reduce manipulation errors based on the range of movement, which is suitable from the viewpoint of safety.

As described in FIGS. 14 and 18, the master-side device 1 as a surgery assisting device may include a driver (the master-side driver 90) configured to feed back a tactile sensation detected in the slave-side device 2 via the first manipulator (the grasping part 71 or the grasping structure 59), and the controller may control the drive unit to move the first manipulator to the position (e.g., approximately the center of the range of movement).

This configuration and operation makes it possible to gently guide the surgeon's hand and thus to move the first manipulator to a suitable manipulation position without damaging the surgeon's hand.

As described with reference to FIG. 12 and others, when the second manipulator (the clutch 76) is manipulated, the controller (the master-side controller 93) of the master-side device 1 as a surgery assisting device may remove the linkage between the first manipulator (the grasping part 71 or the grasping structure 59) and the movable part (a movable part of the arm 22, a movable part of the surgical instrument holding device 23, a movable part of the surgical instrument 24, that is, the slave-side driven structure 100).

This configuration and operation makes it possible to execute the processing of moving the first manipulator to the position after the linkage between the master-side device 1 and the slave-side device 2 is removed, which is suitable in terms of safety.

As described above, the first manipulator (the grasping part 71 or the grasping structure 59) of the master-side device 1 as a surgery assisting device may be a manipulator configured to move the movable part (a movable part of the arm 22, a movable part of the surgical instrument holding device 23, and a movable part of the surgical instrument 24, that is, the slave-side driven structure 100) in axial directions of three axes (a turning axis in the pan direction, a turning axis in the pitch direction, and a turning axis in the roll direction) that are perpendicular to one another, and the position may be a center position of each range of movement in the axial directions of the three axes.

Specifically, the axial directions of the three axes are the front and back direction (the axial direction of the surgical instrument 24), the left and right direction (for forceps as the surgical instrument 24, the direction in which the distal end pieces 83 of the forceps are arranged side by side), and the up and down direction (the direction perpendicular to both the front and back direction and the left and right direction) when looking at the distal end part of the surgical instrument 24 in a direction in which the axis of the surgical instrument 24 extends. Then, the distal end part of the surgical instrument 24 is translated and moved to the center position of each range of movement so that the attitude of the distal end part does not change.

As described with reference to FIG. 17 and others, the controller (the master-side controller 93) of the master-side device 1 as a surgery assisting device may select whether or not to allow movement to the position in response to an operation input (e.g., an input operation to the neutral assistance button Btn1 on the setting screen G1a). In some embodiments, the operation input may be predetermined.

This configuration and operation allows the surgeon to switch on/off the neutral assistance function according to the surgeon's preferences. Therefore, even in a case where a plurality of surgeons use the master-side device 1, different settings can be made for each surgeon, making it possible to improve convenience.

As described in the modification example, the first manipulator (the grasping part 71 or the grasping structure 59) of the master-side device 1 as a surgery assisting device may be a manipulator configured to turn the movable part (a movable part of the arm 22, a movable part of the surgical instrument holding device 23, and a movable part of the surgical instrument 24, that is, the slave-side driven structure) in directions around three axes that are perpendicular to one another, and the position may be a central angular position of each range of movement in the directions around the three axes.

As a result, when the neutral assistance function is used, not only translational manipulations but also turning manipulations are automatically adjusted to suitable positions, allowing surgery to be resumed at the optimal hand position and hand attitude.

As described with reference to FIG. 12 and others, the master-side device 1 as a surgery assisting device may include a continuous portion (the first coupling part 70) that is continuous with the grasping part 71 of the first manipulator, and the second manipulator (the clutch 76) may be provided in either the grasping part or the continuous portion.

This configuration and operation allows the surgeon to manipulate the second manipulator while performing translational movement and turning motion on the distal end part of the surgical instrument 24 while grasping the grasping part 71, making it possible to improve ease of manipulation.

As described with reference to FIGS. 14 and 17 and others, the first manipulator (the grasping part 71 or the grasping structure 59) of the master-side device 1 as a surgery assisting device may include a right first manipulator (the grasping portion 71 or the grasping structure 59 of the right manipulator 4R) to be manipulated by the right hand of a user (surgeon or operator) and a left first manipulator (the grasping portion 71 or the grasping structure 59 of the left manipulator 4L) to be manipulated by the left hand of the user (surgeon), the second right manipulator may include a right second manipulator (the clutch 76 of the right manipulator 4R) corresponding to the right first manipulator and a left second manipulator (the clutch 76 of the left manipulator 4L) corresponding to the left first manipulator, and the control unit (the master-side controller 93) may perform processing of moving positions of the left first manipulator and the right first manipulator to respective positions when the right second manipulator and the left second manipulator are manipulated together.

Providing a configuration to start the neutral assistance function in response to simultaneous manipulation of the clutches 76 on both hands makes it possible to prevent wrong manipulation.

As described with reference to FIGS. 14 and 17 and others, the slave-side device 2 may include a right movable part (a movable part of the arm 22R, a movable part of the surgical instrument holding device 23 for the surgical instrument 24R, and a movable part of the surgical instrument 24R) corresponding to the right first manipulator (the grasping part 71 or the grasping structure 59 of the right manipulator 4R) and a left movable part (a movable part of the arm 22L, a movable part of the surgical instrument holding device 23 for the surgical instrument 24L, a movable part of the surgical instrument 24L) corresponding to the left first manipulator (the grasping part 71 or the grasping structure 59 of the left manipulator 4L), and the controller (the master-side control part 93) may remove the linkage between the right first manipulator and the right movable part when the right second manipulator is manipulated, and remove the linkage between the left first manipulator and the left movable part when the left second manipulator is manipulated.

As a result, when the neutral assistance function is started in response to simultaneous manipulation of the clutches 76 on both hands, the linkage between the right first manipulator and the right movable part and the linkage between the left first manipulator and the left movable part are both removed (suspended) inevitably, which is suitable in terms of safety.

A surgery assisting device in the surgery assisting system S described above is a master-side device 1 for remotely manipulating a slave-side device 2, and may include: a first manipulator (the grasping part 71 or the grasping structure 59) having a range of movement and used to manipulate a movable part (a movable part of the arm 22, a movable part of the surgical instrument holding device 23, and a movable part of the surgical instrument 24, that is, the slave-side driven structure) of the slave-side device 2; and a controller (the master-side controller 93) configured to control the movable part in response to a manipulation input to the first manipulator, wherein the controller may perform processing of determining a correspondence relationship between an amount of manipulation of the first manipulator and an amount of movement of the movable part.

This configuration and operation makes it possible to move the corresponding movable part by a large amount by simply moving the first manipulator by a small amount.

Therefore, the surgeon can move the distal end part of the surgical instrument 24 as intended by moving the surgeon's wrist within the range of movement of the wrist, making it possible to improve operability.

Furthermore, the number of times the above-described neutral assistance function is used can be reduced, making it possible to perform a smooth and quick surgery.

As described with reference to FIGS. 14 and 18 and others, a correspondence relationship between an amount of manipulation of the first manipulator (the grasping part 71 or the grasping structure 59) and an amount of movement of the movable part (a movable part of the arm 22, a movable part of the surgical instrument holding device 23, a movable part of the surgical instrument 24, that is, the slave-side driven structure) may be a relationship between an amount of manipulation of the first manipulator (the grasping part 71 or the grasping structure 59) and amounts of movement of turning of the movable part in the pan direction and the pitch direction.

This configuration and operation makes it possible to turn the distal end part of the surgical instrument 24 beyond the range of movement of the surgeon's wrist. Therefore, various manipulations can be performed easily.

As described with reference to FIGS. 14 and 18 and others, a correspondence relationship between an amount of manipulation of the first manipulator (the grasping part 71 or the grasping structure 59) and an amount of movement of the movable part (a movable part of the arm 22, a movable part of the surgical instrument holding device 23, a movable part of the surgical instrument 24, that is, the slave-side driven structure) may be a relationship between an amount of manipulation of the first manipulator and an amount of movement of turning of the movable part in the roll direction.

This configuration and operation allows the distal end part of the surgical instrument 24 to make a motion beyond the range of movement of the surgeon's wrist when the surgeon twists the wrist to turn the distal end part of the surgical instrument 24 in the roll direction.

As described with reference to FIGS. 16 and 18 and others, the controller (the master-side controller 93) of the master-side device 1 as a surgery assisting device in the surgery assisting system S described above may perform display processing that allows a selection of correspondence relationships.

This configuration and operation allows the surgeon to select an amount of manipulation and amounts of movement in the pan direction and the pitch direction. Accordingly, the correspondence relationship can be customized according to the surgeon's preferences, which is suitable in a case where a plurality of surgeons use the master-side device 1.

As described with reference to FIGS. 14, 16, and 18 and others, a display (the second monitor 8) may be included in which an image is displayed by display processing performed by the controller (the master-side controller 93) of the master-side device 1 as a surgery assisting device in the surgery assisting system S, and the controller may display on the display unit a screen (the setting screen G1b) on which a selection field (the ratio selection field Sel4) that allows a selection of correspondence relationships is arranged, and determines a correspondence relationship in response to an operation on the selection field.

This configuration and operation allows the surgeon to perform a selection operation on the ratio selection field Sel4 arranged on the setting screen G1b displayed on the second monitor 8 and thus to change the correspondence relationship. Therefore, change operations are facilitated, and interference with surgery can be suppressed.

As described with reference to FIG. 16 and others, the controller (the master-side controller 93) of the master-side device 1 as a surgery assisting device in the surgery assisting system S may arrange a selection field including an option in which the amount of movement of the movable part (a movable part of the arm 22, a movable part of the surgical instrument holding device 23, a movable part of the surgical instrument 24, that is, the slave-side driven structure) is larger than the amount of manipulation of the first manipulator (the grasping part 71 or the grasping structure 59).

This configuration and operation makes it possible to move the movable part beyond the range of movement of the surgeon's wrist. Therefore, a desired manipulation can be achieved with a single manipulation without removing the linkage between the master-side device 1 and the slave-side device 2, making it possible to improve convenience.

As described with reference to FIG. 16 and others, the controller (the master-side controller 93) of the master-side device 1 as a surgery assisting device in the surgery assisting system S may arrange a selection field including an option in which the amount of movement of the movable part (a movable part of the arm 22, a movable part of the surgical instrument holding device 23, a movable part of the surgical instrument 24, that is, the slave-side driven structure) is smaller than the amount of manipulation of the first manipulator (the grasping part 71 or the grasping structure 59).

This configuration and operation makes it possible to move the movable part by a small amount even when the surgeon's wrist is moved significantly, making it possible to suitably respond to more precise manipulations. Therefore, it is possible to reduce manipulation errors, making it possible to improve surgical safety.

A surgery assisting device in the surgery assisting system S described above is a master-side device 1 for remotely manipulating a slave-side device 2, and may include: a first manipulator (the grasping part 71 or the grasping structure 59) having a range of movement and used to manipulate a movable part (a movable part of the arm 22, a movable part of the surgical instrument holding device 23, and a movable part of the surgical instrument 24, that is, the slave-side driven structure) of the slave-side device 2; and a controller (the master-side controller 93) configured to control the movable part to turn in the roll direction in response to a specific manipulation on the first manipulator, wherein the controller may perform processing of determining where or not to turn the movable part in the roll direction in response to the specific manipulation.

This configuration and operation makes it possible to remove the linkage between the master-side device 1 and the slave-side device 2.

In some embodiments, a configuration is provided in which the linkage can be removed only for translational motion in a direction or turning motion in a direction around an axis, making it possible to provide manipulation modes that meet various needs of the surgeon and thus to improve convenience. In some embodiments, the direction may be predetermined. In some embodiments, the axis may be predetermined.

As described with reference to FIGS. 14, 16, and 19 and others, in the master-side device 1 as a surgery assisting device in the surgery assisting system S, the specific manipulation on the first manipulator (the grasping part 71 or the grasping structure 59) may be a manipulation for turning the movable part (a movable part of the arm 22, a movable part of the surgical instrument holding device 23, a movable part of the surgical instrument 24, that is, the slave-side driven structure) in the roll direction (the roll direction turning manipulation Ro), and the controller (the master-side controller 93) may determine whether or not to turn the movable part in the roll direction (the turning motion Ro′) in response to the specific manipulation in the processing of determining (the processing of determining whether or not to move the movable part in response to a manipulation on the first manipulator).

As a result, when the surgeon twists the wrist to turn the distal end part of the surgical instrument 24 in the roll direction but the desired manipulation is disabled due to the range of movement of the wrist, it is possible to remove the linkage in the roll direction and to return the wrist to an attitude that makes it easy to manipulate accordingly. This configuration and operation makes it possible to provide an environment in which appropriate motions can be made when a large number of turning motions in the roll direction are desired.

As described with reference to FIGS. 14, 16, and 19 and others, the control unit (the master-side controller 93) of the master-side device 1 as a surgery assisting device in the surgery assisting system S may perform display processing that allows a selection of options, and when an option is selected, determine not to turn the movable part in response to the specific manipulation in the processing of determining (the processing of determining whether or not to move the movable part (a movable part of the arm 22, a movable part of the surgical instrument holding device 23, a movable part of the surgical instrument 24, that is, the slave-side driven structure) in response to a manipulation on the first manipulator). In some embodiments, the options may be predetermined.

This configuration and operation allows the surgeon to select, for example, whether or not to perform a turning motion in the roll direction on the slave-side movable part in response to a turning manipulation in the roll direction. Accordingly, the correspondence relationship can be customized according to the surgeon's preferences, which is suitable in a case where a plurality of surgeons use the master-side device 1.

In some embodiments, a configuration is provided that can be implemented by performing an operation on a display unit with a touch panel function, making it possible to achieve intuitive operations, which is advantageous because wrong operations are less likely to occur.

As described with reference to FIGS. 14, 16, and 19 and others, a display (the second monitor 8) may be included in which an image is displayed by display processing performed by the control unit (the master-side controller 93) of the master-side device 1 as a surgery assisting device in the surgery assisting system S, and the controller may display on the display unit a screen (the setting screen G1b) on which a selection operator (the roll clutch button Btn2) for selecting an option is arranged.

As a result, by performing a selection operation on the roll clutch button Btn2 arranged on the setting screen G1b displayed on the second monitor 8, the surgeon can change whether or not to make linkage between the master-side device 1 and the slave-side device 2 for a turning manipulation in the roll direction. Therefore, change operations are facilitated, and interference with surgery can be suppressed.

In some embodiments, a configuration is provided that can be implemented by performing an operation on a display with a touch panel function, making it possible to achieve intuitive operations, which is advantageous because wrong operations are less likely to occur.

As described with reference to FIG. 16 and others, as options, only a first option (Off option) for selecting to perform turning of the movable part (a movable part of the arm 22, a movable part of the surgical instrument holding device 23, a movable part of the surgical instrument 24, that is, the slave-side driver 100) in the roll direction and a second option (On option) for selecting not to perform the turning may be set, and the selection operator may be a button operator (the roll clutch button Btn2) that switches between a state in which the first option is selected and a state in which the second option is selected each time the slave-side driver 100 is operated.

This configuration and operation makes it easy to change settings. Therefore, settings can be changed without interfering with the progress of the surgery.

Note that the various examples described above can be combined in any way.

What is claimed is:

1. A master-side device for remotely manipulating a slave-side device, the master-side device comprising:
a first manipulator used to manipulate a movable part of the slave-side device; and
control processing circuitry configured to at least:
perform determining processing of determining whether or not to turn the movable part in a roll direction, and
based on determining to turn the movable part in the roll direction, control the movable part to turn in the roll direction in response to a specific manipulation of the first manipulator.

2. The master-side device according to claim 1, wherein the determining processing comprises:
performing display processing that allows a selection of one or more options, and
based on an option of the one or more options being selected, determining not to turn the movable part in response to the specific manipulation.

3. The master-side device according to claim 2, comprising a display,
wherein the display processing displays a screen on the display on which a selection operator for selecting the option is arranged.

4. The master-side device according to claim 3, wherein:
the one or more options include only a first option for selecting to perform turning of the movable part in the roll direction and a second option for selecting not to perform the turning of the movable part, and
the selection operator comprises a button that switches between a state in which the first option is selected and a state in which the second option is selected each time the button is operated.

5. The master-side device according to claim 4, wherein the first manipulator has a linkage to the slave-side device that links the master-side device to the slave-side device and allows the first manipulator to manipulate the movable part of the slave-side device, and
wherein the control processing circuitry is configured to switch on and off the linkage based on the button.

6. The master-side device according to claim 1, the first manipulator has a linkage to the slave-side device that links the master-side device to the slave-side device and allows the first manipulator to manipulate the movable part of the slave-side device, and
wherein based on determining to turn the movable part in the roll direction, control the movable part to turn in the roll direction in response to the specific manipulation of the first manipulator, and based on determining not to turn the movable part in the roll direction, control the master-side device to switch off the linkage.

7. A master-side device for remotely manipulating a slave-side device, the master-side device comprising:
a first manipulator having a range of movement in a roll direction, the first manipulator manipulating a movable part of the slave-side device; and
control processing circuitry configured to at least:
determine whether or not to turn the movable part in the roll direction, and
based on determining to turn the movable part in the roll direction, control the movable part to turn in the roll direction in response to a roll manipulation of the first manipulator.

8. The master-side device according to claim 7, further comprising a display,
wherein the control processing circuitry controls the display to display one or more options on the display, and
based on a selection of an option of the one or more options, the control processing circuitry determines whether or not to turn the movable part in the roll direction in response to the roll manipulation.

9. The master-side device according to claim 8, wherein the selection is performed by a button displayed on the display.

10. The master-side device according to claim 8, wherein:
the one or more options include only a first option for selecting to perform turning of the movable part in the roll direction and a second option for selecting not to perform the turning of the movable part, and
the control processing circuitry controls the display to display a button that switches between the first option and the second option each time the button is operated.

11. The master-side device according to claim 7, further comprising a display,
wherein the control processing circuitry controls the display to display a button that toggles between controlling to turn the movable part and not controlling to turn the movable part, and the control processing circuitry determines whether or not to turn the movable part based on a state of the button.

12. The master-side device according to claim 11, wherein the first manipulator has a linkage to the slave-side device that links the master-side device to the slave-side device and allows the first manipulator to manipulate the movable part of the slave-side device, and
wherein the control processing circuitry is configured to switch on and off the linkage based on the state of the button.

13. The master-side device according to claim 7, the first manipulator has a linkage to the slave-side device that links the master-side device to the slave-side device and allows the first manipulator to manipulate the movable part of the slave-side device, and
wherein based on determining to turn the movable part in the roll direction, the control processing circuitry controls the movable part to turn in the roll direction in response to the roll manipulation of the first manipulator, and based on determining not to turn the movable part in the roll direction, the control processing circuitry controls the master-side device to switch off the linkage such that when the first manipulator is turned in the roll direction in response to the roll manipulation the slave-side device does not turn the movable part in the roll direction.

14. A master-side device for remotely manipulating a slave-side device, the master-side device comprising:
a display;
a first manipulator that has a linkage with the slave-side device, the linkage allowing the first manipulator to manipulate a movable part of the slave-side device; and
control processing circuitry configured to at least:
control the display to display a button that toggles between controlling to turn the movable part in a roll direction and not controlling to turn the movable part in the roll direction in response to a roll manipulation of the first manipulator, and
based on a first state of the button, maintain the linkage such that the movable part of the slave-side device is turned in response to the roll manipulation of the first manipulator, and based on a second state of the button, switch off the linkage such that when the first manipulator is turned in the roll direction in response to the roll manipulation the movable part of the slave-side device is not turned in the roll direction.
